SEQUENCE LISTING (136 sequences)

SEQ ID NO: 1 - protein, 13 aa, synthetic construct
SFHDDSDEDLLHI

SEQ ID NO: 2 - protein, 8 aa, synthetic construct
DDSDEDLL

SEQ ID NO: 3 - protein, 13 aa, synthetic construct
EESEERDDHLLPM

SEQ ID NO: 4 - protein, 10 aa, synthetic construct
SYKYSKVNKE

SEQ ID NO: 5 - protein, 6 aa, synthetic construct
YKYSKV

SEQ ID NO: 6 - protein, 10 aa, synthetic construct
PAAYRGVGDD

SEQ ID NO: 7 - protein, 11 aa, synthetic construct
RKRSHAGYQTI

SEQ ID NO: 8 - protein, 13 aa, synthetic construct
ASSGLDDLDLLGK

SEQ ID NO: 9 - protein, 13 aa, synthetic construct
VQNPSADRNLLDL

SEQ ID NO: 10 - protein, 13 aa, synthetic construct
ASVSLLDDELMSL

SEQ ID NO: 11 - protein, 16 aa, synthetic construct
RRRASVSLLDDELMSL

SEQ ID NO: 12 - protein, 10 aa, synthetic construct
ASVSLLDDEL

SEQ ID NO: 13 - protein, 13 aa, synthetic construct
NALSWLDEELLCL

SEQ ID NO: 14 - protein, 8 aa, synthetic construct
SDEDLLHI

SEQ ID NO: 15 - protein, 11 aa, synthetic construct
RRRSDEDLLHI

SEQ ID NO: 16 - protein, 13 aa, synthetic construct
RRLRKSDEDLLHI

SEQ ID NO: 17 - protein, 13 aa, synthetic construct
RRRRKSDEDLLHI

SEQ ID NO: 18 - protein, 16 aa, synthetic construct
RRRSFHDDSDEDLLHI

SEQ ID NO: 19 - protein, 18 aa, synthetic construct
RRLRKSFHDDSDEDLLHI

SEQ ID NO: 20 - protein, 18 aa, synthetic construct
RRRRKSFHDDSDEDLLHI

SEQ ID NO: 21 - protein, 16 aa, synthetic construct
RRRRKRKRSHAGYQTI

SEQ ID NO: 22 - protein, 10 aa, synthetic construct
KHHHAGYEQF

SEQ ID NO: 23 - protein, 14 aa, synthetic construct
RRLRKHHHAGYEQF

SEQ ID NO: 24 - protein, 5 aa, synthetic construct
RRLRK

SEQ ID NO: 25 - protein, 5 aa, synthetic construct
RRRRK

SEQ ID NO: 26 - protein, 11 aa, synthetic construct
YGRKKRRQRRR

SEQ ID NO: 27 - protein, 18 aa, synthetic construct
LLIILRRRIRKQAHAHSK

SEQ ID NO: 28 - protein, 16 aa, synthetic construct
RQIKIWFQNRRMKWKK

SEQ ID NO: 29 - protein, 9 aa, synthetic construct
RRRRRRRRR

SEQ ID NO: 30 - protein, 27 aa, synthetic construct
GWTLNSAGYLLGKINLKALAALAKKIL

SEQ ID NO: 31 - protein, 7 aa, synthetic construct
LPETGGG

SEQ ID NO: 32 - protein, 5 aa, synthetic construct
GGGGG

SEQ ID NO: 33 - protein, 5 aa, synthetic construct
GGGGS

SEQ ID NO: 34 - protein, 10 aa, synthetic construct
GGGGSGGGGS

SEQ ID NO: 35 - protein, 15 aa, synthetic construct
GGGGSGGGGSGGGGS

SEQ ID NO: 36 - protein, 6 aa, synthetic construct
LARLLT

SEQ ID NO: 37 - protein, 17 aa, synthetic construct
RRPPRSPDHKRYFRDKE

SEQ ID NO: 38 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSFHDDSDEDLLHI

SEQ ID NO: 41 - protein, 478 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSFHDDSDEDLLHI

SEQ ID NO: 42 - protein, 247 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 43 - protein, 483 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 44 - protein, 239 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSYKYSKVNKE

SEQ ID NO: 45 - protein, 475 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSYKYSKVNKE

SEQ ID NO: 46 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 247 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 49 - protein, 482 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 50 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 242 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSFHDDSDEDLLHI

SEQ ID NO: 53 - protein, 476 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSFHDDSDEDLLHI

SEQ ID NO: 54 - protein, 247 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 55 - protein, 481 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 56 - protein, 242 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEESEERDDHLLPM

SEQ ID NO: 57 - protein, 476 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEESEERDDHLLPM

SEQ ID NO: 58 - protein, 239 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSYKYSKVNKE

SEQ ID NO: 59 - protein, 473 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSYKYSKVNKE

SEQ ID NO: 60 - protein, 239 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSPAAYRGVGDD

SEQ ID NO: 61 - protein, 473 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSPAAYRGVGDD

SEQ ID NO: 62 - protein, 240 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRKRSHAGYQTI

SEQ ID NO: 63 - protein, 474 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRKRSHAGYQTI

SEQ ID NO: 64 - protein, 242 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASSGLDDLDLLGK

SEQ ID NO: 65 - protein, 476 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSASSGLDDLDLLGK

SEQ ID NO: 66 - protein, 242 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSVQNPSADRNLLDL

SEQ ID NO: 67 - protein, 476 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVQNPSADRNLLDL

SEQ ID NO: 68 - protein, 242 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASVSLLDDELMSL

SEQ ID NO: 69 - protein, 476 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSASVSLLDDELMSL

SEQ ID NO: 70 - protein, 242 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNALSWLDEELLCL

SEQ ID NO: 71 - protein, 476 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNALSWLDEELLCL

SEQ ID NO: 72 - protein, 239 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSKHHHAGYEQF

SEQ ID NO: 73 - protein, 473 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKHHHAGYEQF

SEQ ID NO: 74 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 246 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 77 - protein, 484 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 78 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 452 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 247 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 81 - protein, 485 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 82 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 247 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 85 - protein, 478 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSRRRRKSFHDDSDEDLLHI

SEQ ID NO: 86 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 247 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRRRRKSFHDDSDEDLLHI

SEQ ID NO: 89 - protein, 486 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 90 - protein, 214 aa, synthetic construct; identical to SEQ ID NO: 86

SEQ ID NO: 91 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 210 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 93 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 247 aa, synthetic construct; identical to SEQ ID NO: 88

SEQ ID NO: 95 - protein, 486 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRRRRKSFHDDSDEDLLHI

SEQ ID NO: 96 - protein, 243 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSRRRRKSFHDDSDEDLLHI

SEQ ID NO: 97 - protein, 487 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRRRRKSFHDDSDEDLLHI

SEQ ID NO: 98 - protein, 214 aa, synthetic construct; identical to SEQ ID NO: 38

SEQ ID NO: 99 - protein, 700 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 100 - protein, 239 aa, synthetic construct; identical to SEQ ID NO: 44

SEQ ID NO: 101 - protein, 725 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSSYKYSKVNKE

SEQ ID NO: 102 - protein, 214 aa, synthetic construct; identical to SEQ ID NO: 38

SEQ ID NO: 103 - protein, 700 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEESEERDDHLLPM

SEQ ID NO: 105 - protein, 728 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEESEERDDHLLPM

SEQ ID NO: 106 - protein, 124 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHH

SEQ ID NO: 107 - protein, 144 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGSFHDDSDEDLLHI

SEQ ID NO: 108 - protein, 149 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRRRKSFHDDSDEDLLHI

SEQ ID NO: 109 - protein, 139 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGDDSDEDLL

SEQ ID NO: 110 - protein, 144 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGEESEERDDHLLPM

SEQ ID NO: 111 - protein, 141 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGSYKYSKVNKE

SEQ ID NO: 112 - protein, 137 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGYKYSKV

SEQ ID NO: 113 - protein, 141 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGPAAYRGVGDD

SEQ ID NO: 114 - protein, 142 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRKRSHAGYQTI

SEQ ID NO: 115 - protein, 144 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGASSGLDDLDLLGK

SEQ ID NO: 116 - protein, 144 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGVQNPSADRNLLDL

SEQ ID NO: 117 - protein, 144 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGASVSLLDDELMSL

SEQ ID NO: 118 - protein, 147 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRRASVSLLDDELMSL

SEQ ID NO: 119 - protein, 141 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGASVSLLDDEL

SEQ ID NO: 120 - protein, 144 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGNALSWLDEELLCL

SEQ ID NO: 121 - protein, 139 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGSDEDLLHI

SEQ ID NO: 122 - protein, 142 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRRSDEDLLHI

SEQ ID NO: 123 - protein, 145 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRLRRKSDEDLLHI

SEQ ID NO: 124 - protein, 145 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRRRRKSDEDLLHI

SEQ ID NO: 125 - protein, 147 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRRSFHDDSDEDLLHI

SEQ ID NO: 126 - protein, 149 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRLRKSFHDDSDEDLLHI

SEQ ID NO: 127 - protein, 149 aa, synthetic construct; identical to SEQ ID NO: 108

SEQ ID NO: 128 - protein, 147 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRRRKRKRSHAGYQTI

SEQ ID NO: 129 - protein, 141 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGKHHHAGYEQF

SEQ ID NO: 130 - protein, 145 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHHLPETGGGRRLRKHHHAGYEQF

SEQ ID NO: 131 - protein, 130 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 132 - protein, 148 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHHGGGGSSFHDDSDEDLLHI

SEQ ID NO: 133 - protein, 153 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHHGGGGSRRRRKSFHDDSDEDLLHI

SEQ ID NO: 134 - protein, 19 aa, synthetic construct
LARLLTSFHDDSDEDLLHI

SEQ ID NO: 135 - protein, 24 aa, synthetic construct
LARLLTRRRRKSFHDDSDEDLLHI

SEQ ID NO: 136 - protein, 41 aa, synthetic construct
RRPPRSPDHKRYFRDKEYGRKKRRQRRRSFHDDSDEDLLHI

(19) United States
(12) Patent Application Publication
(10) Pub. No.: US 2026/0274986 A1
(43) Pub. Date: Sep. 17, 2026

(54) USE OF CHIMERA IN TARGETED PROTEIN DEGRADATION TECHNOLOGY

(71) Applicant: SUN YAT-SEN UNIVERSITY, Guangzhou, Guangdong (CN)

(72) Inventors: Xiaoqing CAI, Guangzhou, Guangdong (CN); Xianxing JIANG, Guangzhou, Guangdong (CN)

(21) Appl. No.: 19/469,272

(22) PCT Filed: May 27, 2024
(86) PCT No.: PCT/CN2024/095384

§ 371 Date: Nov. 11, 2025

(30) Foreign Application Priority Data
Mar. 27, 2023 (CN) 202310307108.4
Nov. 16, 2023 (CN) 202311537190.6
Jan. 12, 2024 (CN) 202410054868.3

(51) Int. Cl. C07K 16/32; A61K 38/00; A61K 47/64; C07K 16/28; C12N 15/62

(52) U.S. Cl. CPC C07K 16/32; A61K 38/00; A61K 47/64; C07K 16/2863; C12N 15/625; C07K 2317/41; C07K 2319/06; C07K 2319/95

(57) ABSTRACT

Disclosed is the use of a chimera in a targeted protein degradation technique. The chimera comprises a molecule that selectively binds to a protein of interest and a signal sequence that is directly or indirectly linked to the molecule and mediates the transport of the protein of interest to a lysosome. By constructing a signal-mediated lysosome-targeted chimera, a lysosome-targeted structural unit is used to promote endocytosis, so as to finally realize the enrichment and degradation of the chimera bound protein of interest in the lysosome.

### TECHNICAL FIELD

The present invention belongs to the fields of molecular biology and biopharmaceutical technology, and relates to the application of a chimera in targeted protein degradation technology.

### BACKGROUND OF THE INVENTION

Targeted protein degradation (TPD) technology is a disruptive technology developed over the past decade that directly clears pathogenic proteins. Currently, most drugs are protein inhibitors that achieve therapeutic effects by binding to pathogenic proteins with high affinity to inhibit their activity. Different from the traditional “inhibitory” drug design approach, TPD technology utilizes the cell's existing protein degradation mechanisms to induce the reduction and depletion of the proteins of interest (POIs), representing the most direct and effective way to regulate protein level and intervene in protein function. TPD technology has huge advantages in degrading “undruggable” targets, prolonging drug action time, avoiding off-target effects and adverse reactions, and overcoming drug resistance etc., thus providing a completely new model for disease treatment and prevention.

The most mature technology in this field currently is Proteolysis-Targeting Chimera (PROTAC) technology, which utilizes the intracellular ubiquitin-proteasome system (UPS). It employs bifunctional molecules to recruit E3 ubiquitin ligase, causing K48-linked polyubiquitination of the POIs, which are then recognized and degraded by intracellular proteasomes. PROTAC technology has developed rapidly and become one of the most promising new drug development technologies. However, PROTAC technology relies on the intracellular ubiquitin-proteasome pathway and can only degrade proteins having intracellular binding domains; while cannot degrade membrane proteins or extracellular proteins lacking such domains, which thus limits the types and scope of targets it can degrade.

The lysosomal pathway is another important protein degradation system within cells besides the UPS, regulating the homeostasis of intracellular and extracellular proteins through autophagy or endocytosis. TPD technologies based on the autophagy pathway developed in recent years include Autophagy-Targeting Chimera (AUTAC), Autophagosome-Tethering Compound (ATTEC), and degraders based on Chaperone-Mediated Autophagy (CMA). Among them, AUTAC and ATTEC induce phagophores to envelop the POIs via bifunctional small molecules, subsequently forming autophagosomes, ultimately leading to degradation of POIs. CMA-based degraders utilize a KFERQ peptide motif recognized by the chaperone heat shock protein HSP70, promoting the formation of complexes between the POIs and HSP70 and inducing chaperone-mediated autophagy to achieve the degradation of POIs. Overall, autophagy-based TPD technologies are an important supplement to PROTAC. However, such degradation strategies rely on intracellular autophagy induction signals and are still limited to degrade intracellular proteins of interest, being ineffective against secreted proteins or membrane proteins.

Lysosome-targeting technology based on the endocytosis pathway is an emerging technology developed in the last two years. It promotes the transport of POIs to lysosomes through endocytosis, thereby enabling the degradation of membrane proteins and secreted proteins. Technologies currently developed in this field include Lysosome-Targeting Chimera (LYTAC), Antibody-based TAC (AbTAC), Cytokine-Receptor-Targeting Chimera (KineTAC), and Integrin-Facilitated Lysosomal Degradation (IFLD) strategies. The aforementioned lysosome-targeting degradation technologies based on endocytosis primarily utilize specific Lysosome-Targeting Receptors (LTRs) to achieve lysosome targeting, such as the Cation-Independent Mannose-6-Phosphate Receptor (CI-M6PR), Asialoglycoprotein Receptor (ASGPR), transmembrane E3 ligase (RNF43), cytokine receptor (CXCR7), or integrin; therefore, they are severely limited by the expression levels of these LTRs in different tissues or cell types. Furthermore, it is currently unclear whether over-activating the lysosome-targeting function of these LTRs might impair their original biological functions and lead to drug resistance. In summary, although current endocytosis-lysosome pathway-based TPD technologies can achieve degradation of membrane and extracellular proteins, these technologies depend on specific LTRs to function, posing significant constraints on their further development.

There have been no reports prior to the present disclosure on the use of the chimera disclosed by the present disclosure for degrading the POI bound by the chimera. Therefore, the present disclosure seeks to utilize the lysosomal protein sorting mechanism to apply a chimera containing a lysosome-targeting structural unit to the degradation of its target protein, developing a TPD technology based on lysosome-sorting signals, independent of LTRs, which can be used for the degradation of membrane proteins, secreted proteins, or intracellular proteins.

### SUMMARY OF THE INVENTION

The present disclosure aims to provide the application of a chimera in targeted protein degradation technology. Based on lysosome-targeting degradation technology utilizing the lysosomal protein sorting machinery, the present disclosure is to, by constructing a signal-mediated lysosome-targeting chimera to promote endocytosis, ultimately achieve the enrichment and effective degradation of the protein of interest bound by the chimera in lysosomes.

To achieve the above object, the present disclosure provides use of a chimera in targeted protein degradation technology, the chimera comprising:
(1) a molecule selectively binding to a protein of interest; and
(2) a signal sequence mediating the transport of the protein of interest to the lysosome, which is directly or indirectly fused to the molecule.

The present disclosure provides a method for targeted protein degradation comprising administering to a subject an effective amount of a chimera, the chimera comprising:
(1) a molecule selectively binding to a protein of interest; and
(2) a signal sequence mediating the transport of the protein of interest to the lysosome, which is directly or indirectly fused to the molecule.

The present disclosure provides a chimera comprising:
(1) a molecule selectively binding to a protein of interest; and
(2) a signal sequence mediating the transport of the protein of interest to the lysosome, which is directly or indirectly fused to the molecule.

In some embodiments, the signal sequence mediating the transport of the protein of interest to the lysosome comprises at least one lysosome-targeting structural unit, or a combination of the at least one lysosome-targeting structural unit and at least one cell-penetrating peptide.

In some embodiments, the lysosome-targeting structural unit is at least one selected from the group consisting of a lysosome-sorting signal peptide derived from Golgi-localized, gamma-adaptin ear-containing, Auxin response factor-binding (GGA) protein, a lysosome-sorting signal peptide derived from mannose-6-phosphate receptor (MPR), a lysosome-sorting signal peptide derived from lysosome-associated membrane protein (LAMP), and a mutant thereof, in which the mutant is one of the lysosome-sorting signal peptide mutants of the lysosome-sorting signal peptide derived from GGA protein, the lysosome-sorting signal peptide derived from MPR, or the lysosome-sorting signal peptide derived from LAMP formed by the substitution, deletion, and/or addition of one or more amino acids.

In some embodiments, the lysosome-targeting structural unit is at least one selected from the group consisting of the following amino acid sequences: SFHDDSDEDLLHI (SEQ ID NO: 1), DDSDEDLL (SEQ ID NO: 2), EESEERDDHLLPM (SEQ ID NO: 3), SYKYSKVNKE (SEQ ID NO: 4), YKYSKV (SEQ ID NO: 5), PAAYRGVGDD (SEQ ID NO: 6), RKRSHAGYQTI (SEQ ID NO: 7), ASSGLDDLDLLGK (SEQ ID NO: 8), VQNPSADRNLLDL (SEQ ID NO: 9), ASVSLLDDELMSL (SEQ ID NO: 10), RRRASVSLLDDELMSL (SEQ ID NO: 11), ASVSLLDDEL (SEQ ID NO: 12), NALSWLDEELLCL (SEQ ID NO: 13), SDEDLLHI (SEQ ID NO: 14), RRRSDEDLLHI (SEQ ID NO: 15), RRLRKSDEDLLHI (SEQ ID NO: 16), RRRRKSDEDLLHI (SEQ ID NO: 17), RRRSFHDDSDEDLLHI (SEQ ID NO: 18), RRLRKSFHDDSDEDLLHI (SEQ ID NO: 19), RRRRKSFHDDSDEDLLHI (SEQ ID NO: 20), RRRRKRKRSHAGYQTI (SEQ ID NO: 21), KHHHAGYEQF (SEQ ID NO: 22), RRLRKHHHAGYEQF (SEQ ID NO: 23), and a mutated amino acid sequence of any one of the amino acid sequences of SEQ ID NOs: 1-23 formed by the substitution, deletion, and/or addition of one or more amino acids.

In some embodiments, the lysosome-targeting structural unit is at least one selected from the group consisting of SEQ ID NOs: 1-23.

In some embodiments, the cell-penetrating peptide is at least one selected from the group consisting of the following amino acid sequences: RRR, RRLRK (SEQ ID NO: 24), RRRRK (SEQ ID NO: 25), YGRKKRRQRRR (SEQ ID NO: 26), LLIILRRRIRKQAHAHSK (SEQ ID NO: 27), RQIKIWFQNRRMKWKK (SEQ ID NO: 28), RRRRRRRRR (SEQ ID NO: 29) and GWTLNSAGYLLGKINLKALAALAKKIL (SEQ ID NO: 30).

In some embodiments, the signal sequence mediating the transport of the protein of interest to the lysosome is indirectly fused to the molecule selectively binding to the protein of interest via a first linker peptide or a first chemical linker.

In some embodiments, the lysosome-targeting structural unit is directly fused to the cell-penetrating peptide, or fused to the cell-penetrating peptide via a second linker peptide or a second chemical linker.

In some embodiments, the amino acid sequence of the first or second linker peptide is independently (Leu-Pro-Glu-Thr)ₓ-(Gly_{y1}-Ser_{y2})_{z}, in which x=0 or 1, y1=3, 4, or 5, y2=0 or 1, and z=1, 2, or 3.

In some embodiments, the first or second linker peptide is at least one independently selected from the group consisting of the following amino acid sequences: Leu-Pro-Glu-Thr-Gly-Gly-Gly (SEQ ID NO: 31), Gly-Gly-Gly, Gly-Gly-Gly-Gly-Gly (SEQ ID NO: 32), Gly-Gly-Gly-Gly-Ser (SEQ ID NO: 33), Gly-Gly-Gly-Gly-Ser-Gly-Gly-Gly-Gly-Ser (SEQ ID NO: 34), and Gly-Gly-Gly-Gly-Ser-Gly-Gly-Gly-Gly-Ser-Gly-Gly-Gly-Gly-Ser (SEQ ID NO: 35).

In some embodiments, the molecule selectively binding to the protein of interest comprises a polypeptide, a protein, a nucleic acid, a nanoparticle, or a small molecule compound. In some embodiments, the polypeptide is glycosylated or non-glycosylated, or consists of natural amino acids and/or unnatural amino acids.

In some embodiments, the signal sequence mediating the transport of the protein of interest to the lysosome is fused to the polypeptide at at least one of the C-terminus of the polypeptide, the N-terminus of the polypeptide, and an amino acid side chain group of the polypeptide. In some embodiments, the amino acid side chain group comprises a side chain group of a natural amino acid or a side chain group of an unnatural amino acid. In some embodiments, the side chain group of a natural amino acid comprises an amino group and a thiol group; and/or the side chain group of an unnatural amino acid comprises an azido group, alkyne group, aldehyde group, ketone group, fluorosulfonate, chloro group, bromo group, or iodo group.

In some embodiments, the protein is an antibody. In some embodiments, the antibody comprises a polyclonal antibody, a monoclonal antibody, a monospecific antibody, a bispecific antibody, a multispecific antibody, a chimeric antibody, a humanized antibody, a human antibody, or an antigen-binding fragment. In some embodiments, the antigen-binding fragment is a Fab, a Fab′, a F(ab′)₂, a Fv, a dsFv, a scFv, a sc(Fv)₂, or a VHH.

In some embodiments, the signal sequence mediating the transport of the protein of interest to the lysosome is fused to the protein at at least one of the C-terminus of the heavy chain of the antibody, the N-terminus of the heavy chain of the antibody, the C-terminus of the light chain of the antibody, the N-terminus of the light chain of the antibody, an amino acid side chain group of the antibody, the C-terminus of the antigen-binding fragment, the N-terminus of the antigen-binding fragment, and an amino acid side chain group of the antigen-binding fragment.

In some embodiments, the signal sequence mediating the transport of the protein of interest to the lysosome is fused to the molecule selectively binding to the protein of interest by a method comprising at least one of chemical conjugation, enzymatic catalysis, and genetic recombination.

In some embodiments, the protein of interest is at least one of a cell surface protein, an intracellular protein, and an extracellular protein.

In some embodiments, the protein of interest is at least one of a disease-associated protein of interest and an immune checkpoint molecule.

In some embodiments, the disease comprises at least one of a cancer, an inflammation-related disease, an immune-related disease, a viral infection, a metabolic disease, and a neurodegenerative disease.

In some embodiments, the protein of interest is at least one of an internalizable or non-internalizable receptor, a ligand of a receptor, a cytokine, a hormone, a secreted protein, an antibody, a protein carrier, and an enzyme.

In some embodiments, the chimera can be used for targeted protein degradation.

In some embodiments, the present disclosure also provides a nucleic acid molecule comprising a nucleotide sequence encoding the chimera according to any one of the preceding embodiments.

The present disclosure develops the application of a chimera in targeted protein degradation technology. The chimera provided by the present disclosure comprises a molecule selectively binding to a protein of interest and a signal sequence mediating the transport of the protein of interest to the lysosome. This signal sequence mediating the transport of the protein of interest to the lysosome comprises at least one lysosome-targeting structural unit, or a combination of at least one lysosome-targeting structural unit and at least one cell-penetrating peptide. The lysosome-targeting structural unit is a human lysosome-sorting signal peptide or a mutant obtained by using a human lysosome-sorting signal peptide as a template. The chimera provided by the present disclosure utilizes the lysosomal protein sorting mechanism, where the lysosome-targeting structural unit triggers endocytosis, ultimately leading to the enrichment and degradation of the protein of interest bound by the chimera within lysosomes. The chimera provided by the present disclosure can achieve effective degradation and regulation of proteins of interest in vitro and/or in vivo, and can be used for knocking down or degrading proteins of interest in vivo and/or in vitro, as well as for the prevention and treatment of diseases such as cancers, metabolic diseases, and chronic diseases.

### BRIEF DESCRIPTION OF THE DRAWINGS

FIG. 1 displays Western blot images showing the degradation of the cell surface membrane protein HER2 via 24 different chimeras (Nb1 to Nb24) in Example 2 of the present disclosure.

FIG. 2 displays Western blot images showing the degradation of the membrane protein HER2 on three different tumor cells (SKBR3 human breast adenocarcinoma cells, BT474 human breast cancer cells, and SKOV3 human ovarian cancer cells) via 6 different chimeras (Ab1 to Ab6) in Example 2 of the present disclosure.

FIGS. 3A and 3B display plots showing the flow cytometry analysis and quantification results of the degradation of HER2 via chimeras Ab3, Ab6, and Tz-P4 in Example 2 of the present disclosure.

FIGS. 4A and 4B display confocal laser scanning microscopy images showing the degradation of HER2 via chimeras Ab3, Ab6, and Tz-P4 in Example 2 of the present disclosure.

FIG. 5 displays Western blot images showing the HER2 levels after cycloheximide inhibition and incubation with chimeras (Ab1, Ab3, Ab4, and Ab6) in Example 2 of the present disclosure.

FIGS. 6A, 6B, and 6C display Western blot images showing the degradation of EGFR on A431 human epidermoid carcinoma cells and/or A549 human lung adenocarcinoma cells via three different types of chimeras in Example 3 of the present disclosure: full-length antibody based (Ctx-C3-P1), nanobody based (7D12-P1 and 7D12-C3-P1), and polypeptide based (PEP1-P1 and PEP1-C3-P1).

FIG. 7 displays Western blot images showing the degradation of membrane protein PD-L1 on MDA-MB-231 human breast cancer cells via 11 different chimeras (Az-P1, Az-C3-P1, Az-P3, Az-P4, Az-P6, Az-P7, Az-P8, Az-P9, Az-P10, Az-P13, and Az-P22) in Example 4 of the present disclosure.

FIG. 8 displays confocal laser scanning microscopy images showing the degradation of PD-L1 on MDA-MB-231 cells via two chimeras (Az-P3 and Az-P4) in Example 4 of the present disclosure.

FIG. 9 displays Western blot images showing the degradation of PD-L1 via chimera Az-P4 at different time points in Example 4 of the present disclosure.

FIG. 10 displays Western blot images showing the degradation of CD20 on Ramos human B lymphocytic lymphoma cells via two full-length antibody-based chimeras (Rtx-C3-P1 and Oftm-C3-P1) in Example 5 of the present disclosure.

FIG. 11 displays Western blot images showing the degradation of CD71 on Jurkat human T lymphocytic leukemia cells via a full-length antibody-based chimera (anti-CD71-C3-P1) in Example 6 of the present disclosure.

FIG. 12 display plots showing the flow cytometry analysis and quantification results of the internalization of the extracellular target VEGF in SKBR3 cells after administration of a chimera (BsAb1-C3-P1) in Example 7 of the present disclosure.

FIG. 13 displays confocal laser scanning microscopy images showing the colocalization of VEGF to lysosomes in SKOV3 cells after administration of two different chimeras (Bv-C3-P1 and BsAb1-C3-P1) in Example 7 of the present disclosure.

FIG. 14 displays Western blot images showing the degradation of VEGF via two types of chimeras in Example 7 of the present disclosure: bispecific antibody based (BsAb1-C3-P1, BsAb2-P4, BsAb3-P3) and monospecific antibody based (Bv-C3-P1).

FIG. 15 displays Western blot images showing the degradation of the intracellular target CDK5 in T24 human bladder cancer cells via a chimera (PEP2-C4-P1) in Example 8 of the present disclosure.

FIG. 16 displays Western blot images showing the HER2 levels in the control and experimental groups after co-incubation with different lysosome inhibitors respectively in Example 9 of the present disclosure.

FIG. 17 displays electron microscopy images showing lysosomes and lysosome-related organelles in cells of the control and experimental groups after administration of chimeras in Example 9 of the present disclosure.

FIG. 18 displays Western blot images showing the levels of protein markers associated with the endocytosis-lysosome pathway in the control and experimental groups after administration of chimeras in Example 9 of the present disclosure.

FIGS. 19A and 19B display plots showing the flow cytometry analysis and quantification results of the chimera-promoted tumor cell apoptosis in Example 10 of the present disclosure.

FIG. 20 displays confocal laser scanning microscopy images showing the chimera-inhibited tumor cell proliferation in Example 10 of the present disclosure.

FIGS. 21A and 21B display a graph showing the tumor volume statistics of mice post-modeling and photographs showing excised tumor tissues from mice in Example 11 of the present disclosure.

FIG. 22 displays Western blot images showing the target levels in excised tumor tissues from mice in Example 11 of the present disclosure.

FIG. 23 displays a plot showing the QPCR statistics of excised tumor tissues from mice in Example 11 of the present disclosure.

FIG. 24 displays images showing the TUNEL staining of excised tumor tissues from mice in Example 11 of the present disclosure.

### DETAILED DESCRIPTION OF PREFERRED EMBODIMENTS

Unless otherwise defined, all technical and scientific terms used herein have the same meanings as commonly understood by those skilled in the technical field of the present application. The terminology used in the description of the present application is for the purpose of describing specific embodiments only and is not intended to limit the present application. The terms “comprise”, “include”, “have”, and any variations thereof in the description and claims of the present application, as well as in the above description of the drawings, are intended to cover a non-exclusive inclusion. The terms “first”, “second”, etc., in the description and claims of the present application or the aforementioned drawings are used to distinguish different objects and not to describe a specific order, nor do they indicate that the objects are identical or different.

Reference to “an embodiment/example” herein means that a particular feature, structure, or characteristic described in connection with the embodiment/example may be included in at least one embodiment/example of the present application. The appearance of the phrase in various places in the description is not necessarily all referring to the same embodiment/example, nor is it an exclusive or alternative embodiment/example separate from other embodiments/examples. Those skilled in the art will understand explicitly and implicitly that the embodiments/examples described herein may be combined with other embodiments/examples.

To enable those skilled in the art to better understand the solutions of the invention, the implementation schemes of the invention will be described in detail below with reference to the Examples. However, those skilled in the art will understand that the following Examples are provided only to illustrate the invention and should not be construed as limiting the scope thereof.

In the Examples, where no specific conditions are specified, conventional conditions or the conditions recommended by the manufacturers shall be followed. For reagents or instruments where the manufacturers are not specified, they are conventional products available commercially.

The present disclosure provides the use of a chimera in targeted protein degradation technology, a method for targeted protein degradation, and a chimera and its nucleic acid molecule. The chimera comprises a molecule selectively binding to a protein of interest, and a signal sequence mediating the transport of the protein of interest to the lysosome, which is directly or indirectly fused to the molecule.

The chimera provided by the present disclosure is a signal-mediated lysosome-targeting chimera capable of targeting a protein of interest and mediating the transport of the protein of interest to the lysosome. The chimera is able to facilitate the specific degradation of the protein of interest within lysosomes by mediating the transport of the protein of interest to lysosomes via the signal sequence. In other words, the chimera can promote endocytosis, and ultimately achieve the enrichment and effective degradation of the protein of interest bound by the chimera within lysosomes.

In some embodiments, the chimera consists essentially of the following two parts: (1) a molecule selectively binding to a protein of interest; and (2) a signal sequence mediating the transport of the protein of interest to the lysosome, which is directly or indirectly fused to the molecule.

In some embodiments, the chimera may further comprise a flexible linker peptide (e.g., (GGGGS)ₙ, (GGGS)₃), a rigid linker peptide (e.g., (EAAAK)ₙ, A(EAAAK)₄, ALEA(EAAAK)₄A), an in vivo cleavable peptide linker (e.g., Val-Cit, VA, GGFG), or a chemical linker (e.g., (CH₂CH₂O)ₙ, (CH₂)ₙ), for linking different structural units; or an oligosaccharide; or a polypeptide tag (e.g., Flag-tag, His-tag, HA-tag), an affinity tag (e.g., GST-tag, MBP-tag), or a fluorescent protein tag (e.g., GFP-tag, RFP-tag, mCherry-tag); or any combination thereof, in which n=1-10, and Cit is citrulline.

In some embodiments, in addition to the molecule selectively binding to the protein of interest, the chimera may further comprise any other drug molecules for degrading proteins and/or treating diseases and/or preventing diseases.

In some embodiments, in addition to the molecule selectively binding to the protein of interest, the chimera may not comprise any other drug molecules for degrading proteins and/or treating diseases and/or preventing diseases.

It should be understood that the protein of interest is at least one selected from the group consisting of a cell surface target, an intracellular target, and an extracellular target. Among them, a cell surface target includes, but is not limited to, a growth factor receptor (e.g., a HER family member such as EGFR, HER2, or HER3), an immune checkpoint molecule (e.g., PD-1, PD-L1, CTLA4, TIGIT, or TIM3), a leukocyte differentiation antigen (e.g., CD20, CD22, CD24, CD38, CD71, or CD80), etc. An extracellular target includes, but is not limited to, a growth factor (e.g., VEGF, or EGF), a cytokine (e.g., an interleukin, an interferon, a tumor necrosis factor, or a transforming growth factor), a chemokine and a secreted protein (e.g., α-synuclein), a protein hormone (e.g., insulin), an antibody (e.g., an allergic antibody, or an autoantibody), a protein toxin, a virus, and an infectious particle, etc. An intracellular target includes, but is not limited to, a kinase (e.g., a cyclin-dependent kinase, or a tyrosine kinase), etc.

In some embodiments, the cell surface target is at least one selected from the group consisting of human epidermal growth factor receptor 2 (HER2), epidermal growth factor receptor 1 (EGFR), programmed death-ligand 1 (PD-L1), B-lymphocyte antigen CD20, and transferrin receptor 1 (TfR1 or CD71); the extracellular target is vascular endothelial growth factor (VEGF); and the intracellular target is cyclin-dependent kinase (CDK5).

In some embodiments, the signal sequence mediating the transport of the protein of interest to the lysosome comprises at least one lysosome-targeting structural unit.

In some embodiments, the signal sequence mediating the transport of the protein of interest to the lysosome comprises a combination of at least one lysosome-targeting structural unit and at least one cell-penetrating peptide.

### Lysosome-Targeting Structural Unit

The lysosome-targeting structural unit is used to facilitate the transport of the protein of interest to the lysosome and promote its degradation within the lysosome. In some embodiments, the lysosome-targeting structural unit is at least one selected from the group consisting of a lysosome-sorting signal peptide derived from Golgi-localized, gamma-adaptin ear-containing, Auxin response factor-binding (GGA) protein, a lysosome-sorting signal peptide derived from mannose-6-phosphate receptor (MPR), a lysosome-sorting signal peptide derived from lysosome-associated membrane protein (LAMP), and a mutant thereof, in which the mutant is one of the lysosome-sorting signal peptide mutants of the lysosome-sorting signal peptide derived from GGA protein, the lysosome-sorting signal peptide derived from MPR, or the lysosome-sorting signal peptide derived from LAMP formed by the substitution, deletion, and/or addition of one or more amino acids.

In some examples, the amino acid sequence of the lysosome-targeting structural unit is shown in Table 1. However, it should be noted that the lysosome-targeting structural unit provided by the present disclosure is not restricted to the amino acid sequences listed in Table 1.

TABLE 1
Amino acid sequence of the lysosome-
targeting structural unit

| Lysosome- | | |
|---|---|---|
| targeting | | SEQ |
| structural | Amino acid | ID |
| unit | sequence | NO: |
| P1 | SFHDDSDEDLLHI | 1 |
| P2 | DDSDEDLL | 2 |
| P3 | EESEERDDHLLPM | 3 |
| P4 | SYKYSKVNKE | 4 |
| P5 | YKYSKV | 5 |
| P6 | PAAYRGVGDD | 6 |
| P7 | RKRSHAGYQTI | 7 |
| P8 | ASSGLDDLDLLGK | 8 |
| P9 | VQNPSADRNLLDL | 9 |
| P10 | ASVSLLDDELMSL | 10 |
| P11 | RRRASVSLLDDELMSL | 11 |
| P12 | ASVSLLDDEL | 12 |
| P13 | NALSWLDEELLCL | 13 |
| P14 | SDEDLLHI | 14 |
| P15 | RRRSDEDLLHI | 15 |
| P16 | RRLRKSDEDLLHI | 16 |
| P17 | RRRRKSDEDLLHI | 17 |
| P18 | RRRSFHDDSDEDLLHI | 18 |
| P19 | RRLRKSFHDDSDEDLLHI | 19 |
| P20 | RRRRKSFHDDSDEDLLHI | 20 |
| P21 | RRRRKRKRSHAGYQTI | 21 |
| P22 | KHHHAGYEQF | 22 |
| P23 | RRLRKHHHAGYEQF | 23 |

Among the amino acid sequences shown in Table 1, the amino acid sequences of SEQ ID NOs: 1-6 and SEQ ID NOs: 14-20 correspond to the lysosome-sorting signal peptide of MPR or mutants thereof, the amino acid sequences of SEQ ID NOs: 8-13 correspond to the lysosome-sorting signal peptide of GGA or mutants thereof, and the amino acid sequences of SEQ ID NO: 7 and SEQ ID NOs: 21-23 correspond to the lysosome-sorting signal peptide of LAMP or mutants thereof.

### Cell-Penetrating Peptide

Cell-penetrating peptide possesses cell membrane-penetrating activity and is a polypeptide capable of facilitating the efficient passage of proteins across the cell membrane into the interior of the cell. In some examples, the amino acid sequence of the cell-penetrating peptide is shown in Table 2. However, it should be noted that the cell-penetrating peptide provided by the present disclosure is not restricted to the amino acid sequences listed in Table 2.

TABLE 2
Amino acid sequence of the cell-penetrating peptide

| Cell- | | SEQ |
|---|---|---|
| penetrating | Amino acid | ID |
| peptide | sequence | NO: |
| C1 | RRR | — |
| C2 | RRLRK | 24 |
| C3 | RRRRK | 25 |
| C4 | YGRKKRRQRRR | 26 |
| C5 | LLIILRRRIRKQAHAHSK | 27 |
| C6 | RQIKIWFQNRRMKWKK | 28 |
| C7 | RRRRRRRRR | 29 |
| C8 | GWTLNSAGYLLGKINLKALAALAKKIL | 30 |

In some embodiments, the signal sequence mediating the transport of the protein of interest to the lysosome is indirectly fused to the molecule selectively binding to the protein of interest via a first linker peptide or a first chemical linker.

In some embodiments, the lysosome-targeting structural unit is directly fused to the cell-penetrating peptide, or fused to the cell-penetrating peptide via a second linker peptide or a second chemical linker.

In some embodiments, the amino acid sequence of the first or second linker peptide is independently (Leu-Pro-Glu-Thr)ₓ-(Gly_{y1}-Ser_{y2})_{z}, in which x=0 or 1, y1=3, 4, or 5, y2=0 or 1, and z=1, 2, or 3. In some examples, the amino acid sequence of the linker peptide is shown in Table 3. However, it should be noted that the linker peptide provided by the present disclosure is not restricted to the amino acid sequences listed in Table 3.

TABLE 3
Amino acid sequence of the linker peptide

| Linker peptide | Amino acid sequence | SEQ ID NO: |
|---|---|---|
| Linker1 | LPETGGG | 31 |
| Linker2 | GGG | — |
| Linker3 | GGGGG | 32 |
| Linker4 | GGGGS | 33 |
| Linker5 | GGGGSGGGGS | 34 |
| Linker6 | GGGGGGGGSGGGGS | 35 |

In some embodiments, the structure of the first or second chemical linker is independently any one or combination of those shown in Table 4, in which n is an integer from 1 to 20. However, it should be noted that the chemical linker provided by the present disclosure includes, but is not limited to the structures shown in Table 4, provided it is capable of effectively linking the lysosome-targeting structural unit and the cell-penetrating peptide.

TABLE 4
Structure of the chemical linker

| Chemical linker | Structure |
|---|---|
| Alkyl chain | 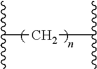 |
| PEG chain | 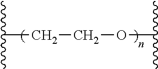 |
| Glycol chain | 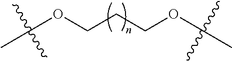 |
| Alkynyl | 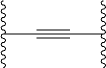 [SMILES: *C#C*] |
| Triazole | 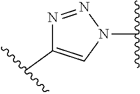 [SMILES: *c1cn(*)nn1] |
| Piperazine | 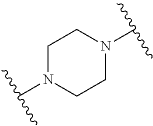 [SMILES: *N1CCN(*)CC1] |
| Piperidine | 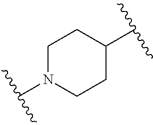 [SMILES: *C1CCN(*)CC1] |

In some embodiments, the molecule selectively binding to the protein of interest comprises a polypeptide, a protein, a nucleic acid, a nanoparticle, or a small molecule compound.

In some embodiments, the polypeptide is glycosylated or non-glycosylated, or consists of natural amino acids and/or unnatural amino acids.

In some embodiments, the signal sequence mediating the transport of the protein of interest to the lysosome is fused to the polypeptide at at least one of the C-terminus of the polypeptide, the N-terminus of the polypeptide, and an amino acid side chain group of the polypeptide.

In some embodiments, the amino acid side chain group comprises a side chain group of a natural amino acid or a side chain group of an unnatural amino acid. The side chain group of a natural amino acid comprises an amino group and a thiol group; and the side chain group of an unnatural amino acid comprises an azido group, alkyne group, aldehyde group, ketone group, fluorosulfonate, chloro group, bromo group, or iodo group.

In some embodiments, the protein is an antibody. In some embodiments, the antibody comprises a polyclonal antibody, a monoclonal antibody, a monospecific antibody, a bispecific antibody, a multispecific antibody, a chimeric antibody, a humanized antibody, a human antibody, or an antigen-binding fragment.

In some embodiments, the antigen-binding fragment is a Fab, a Fab′, a F(ab′)₂, a Fv, a dsFv, a scFv, a sc(Fv)₂, or a VHH.

In some embodiments, the signal sequence is fused to the protein at at least one of the C-terminus of the heavy chain of the antibody, the N-terminus of the heavy chain of the antibody, the C-terminus of the light chain of the antibody, the N-terminus of the light chain of the antibody, an amino acid side chain group of the antibody, the C-terminus of the antigen-binding fragment, the N-terminus of the antigen-binding fragment, and an amino acid side chain group of the antigen-binding fragment.

In some embodiments, the signal sequence mediating the transport of the protein of interest to the lysosome is fused to the molecule by a method comprising at least one of chemical conjugation, enzymatic catalysis, and genetic recombination.

In some embodiments, the protein of interest is a cell surface protein, an intracellular protein, or an extracellular protein.

In some embodiments, the protein of interest further may be a disease-associated protein of interest or an immune checkpoint molecule.

In some embodiments, the disease includes, but is not limited to, a cancer, an inflammation-related disease, an immune-related disease, a viral infection, a metabolic disease, a neurodegenerative disease, etc.

In some embodiments, the protein of interest is an internalizable or non-internalizable receptor, a ligand of a receptor, a cytokine, a hormone, a secreted protein, an antibody, a protein carrier, or an enzyme.

In some embodiments, the signal sequence mediating the transport of the protein of interest to the lysosome is directly fused to the molecule selectively binding to the protein of interest.

In some embodiments, the signal sequence mediating the transport of the protein of interest to the lysosome comprises a combination of at least one lysosome-targeting structural unit and at least one cell-penetrating peptide.

In some embodiments, the lysosome-targeting structural unit is at least one selected from the group consisting of the following amino acid sequences: DDSDEDLL (SEQ ID NO: 2), YKYSKV (SEQ ID NO: 5), and a mutated amino acid sequence of any one of the amino acid sequences of SEQ ID NO: 2 and SEQ ID NO: 5 formed by the substitution, deletion, and/or addition of one or more amino acids.

In some embodiments, the lysosome-targeting structural unit is at least one selected from the group consisting of the following amino acid sequences: DDSDEDLL (SEQ ID NO: 2) and YKYSKV (SEQ ID NO: 5).

In some embodiments, the mutant formed by the substitution, deletion, and/or addition of one or more amino acids in the lysosome-sorting signal peptide exhibits a homology greater than or equal to 70%, 75%, 80%, 81%, 82%, 83%, 84%, 85%, 86%, 87%, 88%, 89%, 90%, 91%, 92%, 93%, 94%, 95%, 96%, 97%, 98%, or 99% compared to the original lysosome-sorting signal peptide.

In some embodiments, the mutant is a mutant of the lysosome-sorting signal peptide formed by the substitution, deletion, and/or addition of one amino acid in the lysosome-sorting signal peptide.

Based on the aforementioned chimera, the present disclosure also provides a method for constructing the chimera, comprising the following steps:
Constructing a plasmid for expressing the chimera;
Expressing, purifying, and characterizing the chimera to obtain the final chimera.

In the method, the step of constructing the plasmid for expressing the chimera comprises:
(1) Identifying a protein of interest and selecting a target molecule selectively binding to the protein of interest;
(2) Fusing a signal sequence mediating the transport of the protein of interest to the lysosome to the corresponding position in the target molecule in a chimeric manner to obtain a target fragment, and then integrating the target fragment into a vector;
(3) Amplifying the target fragment and the vector separately by PCR, requiring that the synthesized target fragment and the vector have an overlapping region, to obtain a PCR product and a plasmid;
(4) Ligating the PCR product into the plasmid using a homologous recombinase to obtain a ligation product;
(5) Transforming the ligation product into DH5α competent cells, plating the cells, and placing the cells into an incubator for overnight culture; and next day, picking single colonies for culture, followed by sequencing to confirm successful construction. Successful construction yields a plasmid for expressing the chimera.

The plasmid for expressing the chimera is then expressed to obtain the expression product; this expression product is then purified to obtain the purified protein. The purified protein is characterized, yielding the final chimera.

It should be noted that in step (3) of constructing the plasmid for expressing the chimera, the target fragment and the vector only need to have an overlapping region, and the size of the overlapping region is not restricted.

In some embodiments, the present disclosure provides the use of the chimera in the preparation of a reagent for targeted protein degradation. In some embodiments, the reagent is a pharmaceutical composition. In some embodiments, the reagent is used for treating and/or preventing a disease associated with the protein of interest (e.g., abnormal expression, or overexpression).

In some embodiments, the method for targeted protein degradation is a method for treating and/or preventing a disease associated with the protein of interest (e.g., abnormal expression, or overexpression).

In some embodiments, the disease associated with the protein of interest includes, but is not limited to, at least one selected from the group consisting of: a cancer, an inflammation-related disease, an immune-related disease, a viral infection, a metabolic disease, and a neurodegenerative disease. In some embodiments, the cancer includes, but is not limited to, at least one selected from the group consisting of: gastric tumor, intestinal tumor, liver tumor, lung tumor, pancreatic tumor, breast tumor, cervical tumor, endometrial tumor, ovarian tumor, prostate tumor, bladder tumor, nasopharyngeal or soft tissue tumor, hematologic or lymphatic tumor, and skin tumor. In some embodiments, the cancer includes, but is not limited to, at least one selected from the group consisting of: breast tumor, ovarian tumor, skin tumor (e.g., epidermal carcinoma), lung tumor (e.g., lung adenocarcinoma), hematologic or lymphatic tumor (e.g., lymphocytic cancer), and bladder tumor.

The embodiments of the invention will be described in detail below with reference to the examples. However, it should be noted that the experimental methods described in this description are applicable for validating the activity and efficacy of all chimeras according to the invention.

### Example 1: Construction of Chimeras

In this Example, molecules of different types and different molecular weight ranges were selected to construct chimeras. Illustratively, the molecules of different types and molecular weight ranges included full-length antibodies (including monospecific and bispecific antibodies), antigen-binding fragments (including nanobodies), and polypeptides. However, it is important to note that the types of molecules include, but are not limited to, the aforementioned types of molecules.

In this Example, full-length antibodies, nanobodies, and polypeptides were used. The monospecific antibody templates used were: the anti-HER2 monoclonal antibody Trastuzumab (hereinafter abbreviated as Tz), the anti-EGFR monoclonal antibody Cetuximab (hereinafter abbreviated as Ctx), the anti-PD-L1 monoclonal antibody Atezolizumab (hereinafter abbreviated as Az), two anti-CD20 monoclonal antibodies Rituximab (hereinafter abbreviated as Rtx) and Ofatumumab (hereinafter abbreviated as Oftm), the anti-CD71 monoclonal antibody (hereinafter abbreviated as anti-CD71), and the anti-VEGF monoclonal antibody Bevacizumab (hereinafter abbreviated as Bv). The bispecific antibody template used was a bispecific antibody against HER2 and VEGF (hereinafter abbreviated as BsAb). The nanobody (also known as VHH) templates used were the anti-HER2 nanobody (5F7) and the anti-EGFR nanobody (7D12). The polypeptides selected for structural engineering were PEP1 (amino acid sequence: LARLLT, shown in SEQ ID NO: 36) having high affinity for EGFR, and PEP2 (amino acid sequence: RRPPRSPDHKRYFRDKE, shown in SEQ ID NO: 37) having high affinity for CDK5. However, it should be noted that the chimeras according to the invention include, but are not limited to, the chimeras listed above.

The lysosome-targeting signal sequence provided by the present disclosure was selectively fused to at least one of the C-terminus of the heavy chain of the antibody, the N-terminus of the heavy chain of the antibody, the C-terminus of the light chain of the antibody, the N-terminus of the light chain of the antibody, an amino acid side chain group of the antibody, the C-terminus of the antigen-binding fragment, the N-terminus of the antigen-binding fragment, an amino acid side chain group of the antigen-binding fragment, the C-terminus of the polypeptide, the N-terminus of the polypeptide, and an amino acid side chain group of the polypeptide.

### 1) Construction of Chimeras Based on the Monoclonal Antibodies

In this Example, a linker peptide, a lysosome-targeting structural unit, and a cell-penetrating peptide were fused to the N-terminus or C-terminus of an antibody via genetic recombination.

### a) Construction of Plasmids for Expressing Chimeras

i. Construction of Plasmids for Expressing Chimeras Targeting HER2

The signal sequence mediating the transport of the protein of interest to the lysosome was fused to Tz at at least one of the C-terminus of the heavy chain of Tz (hereinafter abbreviated as Tz-HC), the N-terminus of the heavy chain of Tz, the C-terminus of the light chain of Tz (hereinafter abbreviated as Tz-LC), and the N-terminus of the light chain of Tz. In this Example, Tz was engineered at genetic level. For illustrative purposes, Linker6 was used as the linker peptide, P1 or P4 was used as the lysosome-targeting structural unit, and C3 was used as the cell-penetrating peptide. Linker6 along with P1 or P4 and/or C3 were fused to the C-termini of the light chain and/or heavy chain of Tz to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing Tz-based chimeras in this Example specifically included the following steps.

First, Linker6 was fused respectively with P1, the combination of P1 and C3 (abbreviated as C3-P1), or P4 to the C-terminus of the heavy chain and the C-terminus of the light chain, to construct fragments Tz-LC-Linker6-P1, Tz-HC-Linker6-P1, Tz-LC-Linker6-C3-P1, Tz-HC-Linker6-C3-P1, Tz-LC-Linker6-P4, and Tz-HC-Linker6-P4. These fragments were respectively integrated into the pcDNA3.1 vector. The expression plasmid for Tz-LC and the expression plasmid for Tz-HC were to be used for expressing the full-length antibody Tz. The expression plasmid for the lysosome-targeting structural unit-fused Tz-LC (Tz-LC-Linker6-P1) and the expression plasmid for the heavy chain (Tz-HC) were to be used for expressing the chimera Ab1. The expression plasmid for the light chain (Tz-LC) and the expression plasmid for the lysosome-targeting structural unit-fused heavy chain (Tz-HC-Linker6-P1) were to be used for expressing the chimera Ab2. The expression plasmid for the lysosome-targeting structural unit-fused light chain (Tz-LC-Linker6-P1) and the expression plasmid for the lysosome-targeting structural unit-fused heavy chain (Tz-HC-Linker6-P1) were to be used for expressing the chimera Ab3. The expression plasmid for the lysosome-targeting structural unit and cell-penetrating peptide-fused light chain (Tz-LC-Linker6-C3-P1) and the expression plasmid for the heavy chain (Tz-HC) were to be used for expressing the chimera Ab4. The expression plasmid for the light chain (Tz-LC) and the expression plasmid for the lysosome-targeting structural unit and cell-penetrating peptide-fused heavy chain (Tz-HC-Linker6-C3-P1) were to be used for expressing the chimera Ab5. The expression plasmid for the lysosome-targeting structural unit and cell-penetrating peptide-fused light chain (Tz-LC-Linker6-C3-P1) and the expression plasmid for the lysosome-targeting structural unit and cell-penetrating peptide-fused heavy chain (Tz-HC-Linker6-C3-P1) were to be used for expressing the chimera Ab6. The expression plasmid for the P4-fused light chain (Tz-LC-Linker6-P4) and the expression plasmid for the P4-fused heavy chain (Tz-HC-Linker6-P4) were to be used for expressing the chimera Tz-P4. The amino acid sequences of chimeras Ab1 to Ab6 and Tz-P4 are shown in Table 5.

TABLE 5
Amino acid sequences of chimeras

| Name | Target fragment | SEQ ID NO: |
|---|---|---|
| Tz | Light chain | 38 |
| | Tz-LC | |
| | Heavy chain | 39 |
| | Tz-HC | |
| Ab1 | Light chain | 40 |
| | Tz-LC-Linker6-P1 | |
| | Heavy chain | 39 |
| | Tz-HC | |
| Ab2 | Light chain | 38 |
| | Tz-LC | |
| | Heavy chain | 41 |
| | Tz-HC-Linker6-P1 | |
| Ab3 | Light chain | 40 |
| | Tz-LC-Linker6-P1 | |
| | Heavy chain | 41 |
| | Tz-HC-Linker6-P1 | |
| Ab4 | Light chain | 42 |
| | Tz-LC-Linker6-C3-P1 | |
| | Heavy chain | 39 |
| | Tz-HC | |
| Ab5 | Light chain | 38 |
| | Tz-LC | |
| | Heavy chain | 43 |
| | Tz-HC-Linker6-C3-P1 | |
| Ab6 | Light chain | 42 |
| | Tz-LC-Linker6-C3-P1 | |
| | Heavy chain | 43 |
| | Tz-HC-Linker6-C3-P1 | |
| Tz-P4 | Light chain | 44 |
| | Tz-LC-Linker6-P4 | |
| | Heavy chain | 45 |
| | Tz-HC-Linker6-P4 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

ii. Construction of Plasmids for Expressing Chimeras Targeting EGFR

The signal sequence mediating the transport of the protein of interest to the lysosome was fused to Ctx at at least one of the C-terminus of the heavy chain of Ctx (hereinafter abbreviated as Ctx-HC), the N-terminus of the heavy chain of Ctx, the C-terminus of the light chain of Ctx (hereinafter abbreviated as Ctx-LC), and the N-terminus of the light chain of Ctx. In this Example, Ctx was engineered at genetic level. For illustrative purposes, Linker6 was used as the linker peptide, P1 was used as the lysosome-targeting structural unit, and C3 was used as the cell-penetrating peptide. P1 was combined with C3 (abbreviated as C3-P1), and Linker6 along with C3-P1 were fused to the C-termini of the light chain and heavy chain of Ctx to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing Ctx-based chimeras in this Example specifically included the following steps.

First, Linker6 was fused respectively with C3-P1 to the C-terminus of the heavy chain and the C-terminus of the light chain, to construct fragments Ctx-LC-Linker6-C3-P1 and Ctx-HC-Linker6-C3-P1. The amino acid sequences of the fragments are shown in Table 6. The fragments Ctx-LC-Linker6-C3-P1 and Ctx-HC-Linker6-C3-P1 were respectively integrated into the pcDNA3.1 vector.

TABLE 6
Amino acid sequences of chimeras

| Name | Target fragment | SEQ ID NO: |
|---|---|---|
| Ctx | Light chain | 46 |
| | Ctx-LC | |
| | Heavy chain | 47 |
| | Ctx-HC | |
| Ctx-C3-P1 | Light chain | 48 |
| | Ctx-LC-Linker6-C3-P1 | |
| | Heavy chain | 49 |
| | Ctx-HC-Linker6-C3-P1 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

iii. Construction of Plasmids for Expressing Chimeras Targeting PD-L1

The signal sequence mediating the transport of the protein of interest to the lysosome was fused to Az at at least one of the C-terminus of the heavy chain of Az (hereinafter abbreviated as Az-HC), the N-terminus of the heavy chain of Az, the C-terminus of the light chain of Az (hereinafter abbreviated as Az-LC), and the N-terminus of the light chain of Az. In this Example, Az was engineered at genetic level. For illustrative purposes, Linker6 was used as the linker peptide, P1 was used as the lysosome-targeting structural unit, and C3 was used as the cell-penetrating peptide. Linker6 along with P1 and/or C3 were fused to the C-termini of the light chain and heavy chain of Az to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing Az-based chimeras in this Example specifically included the following steps.

First, Linker6 was fused respectively with P1, P3, P4, P6, P7, P8, P9, P10, P13, P22 and/or C3 to the C-terminus of the heavy chain and the C-terminus of the light chain, to construct fragments Az-LC-Linker6-P1, Az-HC-Linker6-P1, Az-LC-Linker6-C3-P1, Az-HC-Linker6-C3-P1, Az-LC-Linker6-P3, Az-HC-Linker6-P3, Az-LC-Linker6-P4, Az-HC-Linker6-P4, Az-LC-Linker6-P6, Az-HC-Linker6-P6, Az-LC-Linker6-P7, Az-HC-Linker6-P7, Az-LC-Linker6-P8, Az-HC-Linker6-P8, Az-LC-Linker6-P9, Az-HC-Linker6-P9, Az-LC-Linker6-P10, Az-HC-Linker6-P10, Az-LC-Linker6-P13, Az-HC-Linker6-P13, Az-LC-Linker6-P22 and Az-HC-Linker6-P22. The amino acid sequences of the fragments are shown in Table 7. These fragments were respectively integrated into the pcDNA3.1 vector.

TABLE 7
Amino acid sequences of chimeras

| Name | Target fragment | SEQ ID NO: |
|---|---|---|
| Az | Light chain | 50 |
| | Az-LC | |
| | Heavy chain | 51 |
| | Az-HC | |
| Az-P1 | Light chain | 52 |
| | Az-LC-Linker6-P1 | |
| | Heavy chain | 53 |
| | Az-HC-Linker6-P1 | |
| Az-C3-P1 | Light chain | 54 |
| | Az-LC-Linker6-C3-P1 | |
| | Heavy chain | 55 |
| | Az-HC-Linker6-C3-P1 | |
| Az-P3 | Light chain | 56 |
| | Az-LC-Linker6-P3 | |
| | Heavy chain | 57 |
| | Az-HC-Linker6-P3 | |
| Az-P4 | Light chain | 58 |
| | Az-LC-Linker6-P4 | |
| | Heavy chain | 59 |
| | Az-HC-Linker6-P4 | |
| Az-P6 | Light chain | 60 |
| | Az-LC-Linker6-P6 | |
| | Heavy chain | 61 |
| | Az-HC-Linker6-P6 | |
| Az-P7 | Light chain | 62 |
| | Az-LC-Linker6-P7 | |
| | Heavy chain | 63 |
| | Az-HC-Linker6-P7 | |
| Az-P8 | Light chain | 64 |
| | Az-LC-Linker6-P8 | |
| | Heavy chain | 65 |
| | Az-HC-Linker6-P8 | |
| Az-P9 | Light chain | 66 |
| | Az-LC-Linker6-P9 | |
| | Heavy chain | 67 |
| | Az-HC-Linker6-P9 | |
| Az-P10 | Light chain | 68 |
| | Az-LC-Linker6-P10 | |
| | Heavy chain | 69 |
| | Az-HC-Linker6-P10 | |
| Az-P13 | Light chain | 70 |
| | Az-LC-Linker6-P13 | |
| | Heavy chain | 71 |
| | Az-HC-Linker6-P13 | |
| Az-P22 | Light chain | 72 |
| | Az-LC-Linker6-P22 | |
| | Heavy chain | 73 |
| | Az-HC-Linker6-P22 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

iv. Construction of Plasmids for Expressing Chimeras Targeting CD20

The signal sequence mediating the transport of the protein of interest to the lysosome was fused to Rtx and Oftm respectively at at least one of the C-termini of the heavy chains of Rtx and Oftm (hereinafter abbreviated as Rtx-HC and Oftm-HC), the N-termini of the heavy chains of Rtx and Oftm, the C-termini of the light chains of Rtx and Oftm (hereinafter abbreviated as Rtx-LC and Oftm-LC), and the N-termini of the light chains of Rtx and Oftm. In this Example, Rtx and Oftm were engineered at genetic level. For illustrative purposes, Linker6 was used as the linker peptide, P1 was used as the lysosome-targeting structural unit, and C3 was used as the cell-penetrating peptide. P1 was combined with C3 (abbreviated as C3-P1), and Linker6 along with C3-P1 were fused to the C-termini of the light chains and heavy chains of Rtx and Oftm to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing Rtx-based and Oftm-based chimeras in this Example specifically included the following steps.

First, Linker6 was fused respectively with C3-P1 to the C-termini of the heavy chains and the C-termini of the light chains, to construct fragments Rtx-LC-Linker6-C3-P1, Rtx-HC-Linker6-C3-P1, Oftm-LC-Linker6-C3-P1 and Oftm-HC-Linker6-C3-P1. The amino acid sequences of the fragments are shown in Table 8. These fragments were respectively integrated into the pcDNA3.1 vector.

TABLE 8
Amino acid sequences of chimeras

| Name | Target fragment | SEQ ID NO: |
|---|---|---|
| Rtx | Light chain | 74 |
| | Rtx-LC | |
| | Heavy chain | 75 |
| | Rtx-HC | |
| Rtx-C3-P1 | Light chain | 76 |
| | Rtx-LC-Linker6-C3-P1 | |
| | Heavy chain | 77 |
| | Rtx-HC-Linker6-C3-P1 | |
| Oftm | Light chain | 78 |
| | Oftm-LC | |
| | Heavy chain | 79 |
| | Oftm-HC | |
| Oftm-C3-P1 | Light chain | 80 |
| | Oftm-LC-Linker6-C3-P1 | |
| | Heavy chain | 81 |
| | Oftm-HC-Linker6-C3-P1 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

v. Construction of Plasmids for Expressing Chimeras Targeting CD71

The signal sequence mediating the transport of the protein of interest to the lysosome was fused to the anti-CD71 at at least one of the C-terminus of the heavy chain of the anti-CD71 (hereinafter abbreviated as anti-CD71-HC), the N-terminus of the heavy chain of the anti-CD71, the C-terminus of the light chain of the anti-CD71 (hereinafter abbreviated as anti-CD71-LC), and the N-terminus of the light chain of the anti-CD71. In this Example, the anti-CD71 was engineered at genetic level. For illustrative purposes, Linker6 was used as the linker peptide, P1 was used as the lysosome-targeting structural unit, and C3 was used as the cell-penetrating peptide. P1 was combined with C3 (abbreviated as C3-P1), and Linker6 along with C3-P1 were fused to the C-termini of the light chain and heavy chain of the anti-CD71 to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing anti-CD71-based chimeras in this Example specifically included the following steps.

First, Linker6 was fused respectively with C3-P1 to the C-terminus of the heavy chain and the C-terminus of the light chain, to construct fragments anti-CD71-LC-Linker6-C3-P1 and anti-CD71-HC-Linker6-C3-P1. The amino acid sequences of the fragments are shown in Table 9. These fragments were respectively integrated into the pcDNA3.1 vector.

TABLE 9
Amino acid sequences of chimeras

| Name | Target fragment | SEQ ID NO: |
|---|---|---|
| anti-CD71 | Light chain | 82 |
| | anti-CD71-LC | |
| | Heavy chain | 83 |
| | anti-CD71-HC | |
| anti-CD71-C3-P1 | Light chain | 84 |
| | anti-CD71-LC-Linker6-C3-P1 | |
| | Heavy chain | 85 |
| | anti-CD71-HC-Linker6-C3-P1 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

vi. Construction of Plasmids for Expressing Chimeras Targeting VEGF

The signal sequence mediating the transport of the protein of interest to the lysosome was fused to Bv at at least one of the C-terminus of the heavy chain of Bv (hereinafter abbreviated as Bv-HC), the N-terminus of the heavy chain of Bv, the C-terminus of the light chain of Bv (hereinafter abbreviated as Bv-LC), and the N-terminus of the light chain of Bv. In this Example, Bv was engineered at genetic level. For illustrative purposes, Linker6 was used as the linker peptide, P1 was used as the lysosome-targeting structural unit, and C3 was used as the cell-penetrating peptide. P1 was combined with C3 (abbreviated as C3-P1), and Linker6 along with C3-P1 were fused to the C-termini of the light chain and heavy chain of Bv to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing Bv-based chimeras in this Example specifically included the following steps.

First, Linker6 was fused respectively with C3-P1 to the C-terminus of the heavy chain and the C-terminus of the light chain, to construct fragments Bv-LC-Linker6-C3-P1 and Bv-HC-Linker6-C3-P1. The amino acid sequences of the fragments are shown in Table 10. These fragments were respectively integrated into the pcDNA3.1 vector.

TABLE 10
Amino acid sequences of chimeras

| Name | Target fragment | SEQ ID NO: |
|---|---|---|
| Bv | Light chain | 86 |
| | Bv-LC | |
| | Heavy chain | 87 |
| | Bv-HC | |
| Bv-C3-P1 | Light chain | 88 |
| | Bv-LC-Linker6-C3-P1 | |
| | Heavy chain | 89 |
| | Bv-HC-Linker6-C3-P1 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

vii. Construction of Plasmids for Expressing Chimeras Targeting VEGF and HER2

Bispecific antibodies against HER2 and VEGF (hereinafter abbreviated as BsAbs) were used as the bispecific antibody templates. The anti-VEGF×HER2 bispecific antibodies constructed in the present disclosure comprise three types of BsAbs: the first type (abbreviated as BsAb1) is a KIH-IgG type, consisting of the anti-VEGF heavy chain (BsAb1-HC1) and light chain (BsAb1-LC1) and the anti-HER2 heavy chain (BsAb1-HC2) and light chain (BsAb1-LC2); the second type (abbreviated as BsAb2) has the anti-VEGF scFv fused to the C-terminus of the heavy chain of Tz; and the third type (abbreviated as BsAb3) has the anti-VEGF scFv fused to the N-terminus of the heavy chain of Tz. The signal sequence mediating the transport of the protein of interest to the lysosome was fused to one BsAb respectively at the C-terminus of BsAb-HC and the C-terminus of BsAb-LC.

In this Example, the bispecific antibodies were engineered at genetic level. For illustrative purposes, Linker6 was used as the linker peptide, P1 was used as the lysosome-targeting structural unit, and C3 was used as the cell-penetrating peptide. Linker6 along with P1 and/or C3 were fused to the C-termini of the light chain and heavy chain of the BsAbs to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-termini of the heavy chain and/or light chain. All chimeras thus obtained fall within the scope of the invention.

The construction of plasmids for expressing BsAb1, BsAb2, and BsAb3-based chimeras in this Example specifically included the following steps.

First, fragments BsAb1-HC1-Linker6-C3-P1, BsAb1-HC2-Linker6-C3-P1, BsAb1-LC1-Linker6-C3-P1, BsAb1-HC2-Linker6-C3-P1, BsAb2-HC-Linker6-P4, BsAb2-LC-Linker6-P4, BsAb3-HC-Linker6-P3 and BsAb3-LC-Linker6-P3 were constructed. The amino acid sequences of the fragments are shown in Table 11. These fragments were respectively integrated into the pcDNA3.1 vector.

TABLE 11
Amino acid sequences of chimeras

| Name | Target fragment | SEQ ID NO: |
|---|---|---|
| BsAb1 | Light chain 1 | 90 |
| | BsAb1-LC1 | |
| | Heavy chain 1 | 91 |
| | BsAb1-HC1 | |
| | Light chain 2 | 92 |
| | BsAb1-LC2 | |
| | Heavy chain 2 | 93 |
| | BsAb1-HC2 | |
| BsAb1-C3-P1 | Light chain 1 | 94 |
| | BsAb1-LC1-Linker6-C3-P1 | |
| | Heavy chain 1 | 95 |
| | BsAb1-HC1-Linker6-C3-P1 | |
| | Light chain 2 | 96 |
| | BsAb1-LC2-Linker6-C3-P1 | |
| | Heavy chain 2 | 97 |
| | BsAb1-HC2-Linker6-C3-P1 | |
| BsAb2 | Light chain | 98 |
| | BsAb2-LC | |
| | Heavy chain | 99 |
| | BsAb2-HC | |
| BsAb2-P4 | Light chain | 100 |
| | BsAb2-LC-Linker6-P4 | |
| | Heavy chain | 101 |
| | BsAb2-HC-Linker6-P4 | |
| BsAb3 | Light chain | 102 |
| | BsAb3-LC | |
| | Heavy chain | 103 |
| | BsAb3-HC | |
| BsAb3-P3 | Light chain | 104 |
| | BsAb3-LC-Linker6-P3 | |
| | Heavy chain | 105 |
| | BsAb3-HC-Linker6-P3 | |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

### b) Expression, Purification and Characterization of Chimeras

293 Freestyle human embryonic kidney cells were seeded in a shake flask. When the cell density reached 2×10⁶ cells/mL, transfection was performed using transfection reagent polyethylenimine (PEI). For transfection, two centrifuge tubes were taken, each containing a volume of fresh medium equal to 10% of the final transfection volume. Into one tube, 160 μL of PEI was added, while 80 μg of plasmids (heavy chain:light chain=2:3) was added to the other tube. The tubes were mixed thoroughly, and the mixtures in the tubes were allowed to stand for 5 minutes. Next, the two mixtures were combined by adding the DNA dilution to the PEI dilution, mixed thoroughly, and left to stand for 20 minutes. The mixture obtained was then transferred to the shake flask for transfection, and 5 days later, the culture supernatant was harvested. The harvested supernatant was filtered through a 0.45 μm filter membrane and the protein was purified from the supernatant using a Protein A affinity chromatography column. The purified protein was structurally characterized by SDS-PAGE, HPLC, and MS.

### 2) Construction of Chimeras Based on the Antigen-Binding Fragments

In this example, a genetic recombination approach was employed to fuse the lysosomal targeting structural units and/or the cell-penetrating peptides to the N-terminus or C-terminus of the VHH via linker peptides. Tags such as c-Myc, polyhistidine, GST, MBP, or lipid tags can be introduced at the C-terminus of the VHH to facilitate detection and purification of the expressed product. In this Example, a His₆ tag was used as the protein purification tag.

### a) Construction of Plasmids for Expressing Chimeras

i. Construction of Chimeras Targeting HER2

In this Example, 5F7 was engineered at genetic level. A His₆ tag was fused to the C-terminus of 5F7, to obtain 5F7-His₆. Linker1 was used as the linker peptide, P1 to P23 were used as the lysosome-targeting structural units, and C3 was used as the cell-penetrating peptide. The His₆ purification tag, the linker peptide, one lysosome-targeting structural unit, and/or the cell-penetrating peptide were fused to the C-terminus of 5F7 to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-terminus of 5F7. All chimeras thus obtained fall within the scope of the invention.

The construction of plasmids for expressing 5F7-based chimeras in this Example specifically included the following steps.

First, a His₆ tag was fused to the C-terminus of 5F7 to construct fragment 5F7-His₆; and then the linker peptide Linker1 was fused respectively with one of P1 to P23 and/or the cell-penetrating peptide C3 to the C-terminus of the His₆, to construct target fragments for constructing chimeras. These target fragments were respectively integrated into the pET-28a vector, and plasmids for expressing chimeras Nb1 to Nb24 were obtained. The amino acid sequences of the plasmids for expressing chimeras Nb1 to Nb24 are shown in Table 12.

TABLE 12
Amino acid sequences of chimeras

| Name | Target fragment | SEQ ID NO: |
|---|---|---|
| 5F7 | 5F7-His₆ | 106 |
| Nb1 | 5F7-His₆-Linker1-P1 | 107 |
| Nb2 | 5F7-His₆-Linker1-C3-P1 | 108 |
| Nb3 | 5F7-His₆-Linker1-P2 | 109 |
| Nb4 | 5F7-His₆-Linker1-P3 | 110 |
| Nb5 | 5F7-His₆-Linker1-P4 | 111 |
| Nb6 | 5F7-His₆-Linker1-P5 | 112 |
| Nb7 | 5F7-His₆-Linker1-P6 | 113 |
| Nb8 | 5F7-His₆-Linker1-P7 | 114 |
| Nb9 | 5F7-His₆-Linker1-P8 | 115 |
| Nb10 | 5F7-His₆-Linker1-P9 | 116 |
| Nb11 | 5F7-His₆-Linker1-P10 | 117 |
| Nb12 | 5F7-His₆-Linker1-P11 | 118 |
| Nb13 | 5F7-His₆-Linker1-P12 | 119 |
| Nb14 | 5F7-His₆-Linker1-P13 | 120 |
| Nb15 | 5F7-His₆-Linker1-P14 | 121 |
| Nb16 | 5F7-His₆-Linker1-P15 | 122 |
| Nb17 | 5F7-His₆-Linker1-P16 | 123 |
| Nb18 | 5F7-His₆-Linker1-P17 | 124 |
| Nb19 | 5F7-His₆-Linker1-P18 | 125 |
| Nb20 | 5F7-His₆-Linker1-P19 | 126 |
| Nb21 | 5F7-His₆-Linker1-P20 | 127 |
| Nb22 | 5F7-His₆-Linker1-P21 | 128 |
| Nb23 | 5F7-His₆-Linker1-P22 | 129 |
| Nb24 | 5F7-His₆-Linker1-P23 | 130 |

The target fragments and the vectors were amplified separately by PCR, requiring that the synthesized target fragments and the vectors contain overlapping regions. The PCR products and the plasmid vectors were then ligated using a homologous recombinase. The resulting ligation products were transformed into DH5α competent cells, which were then plated, placed into an incubator and cultured overnight. Next day, single colonies were picked and cultured, followed by sequencing to confirm successful construction.

ii. Construction of Chimeras Targeting EGFR

The signal sequence mediating the transport of the protein of interest to the lysosome was fused to 7D12 at at least one of the C-terminus of 7D12 and the N-terminus of 7D12. In this Example, 7D12 was engineered at genetic level. For illustrative purposes, a His₆ tag was fused to the C-terminus of 7D12, to obtain 7D12-His₆. Linker4 was used as the linker peptide, P1 was used as the lysosome-targeting structural unit, and C3 was used as the cell-penetrating peptide. The His₆ purification tag, the linker peptide, the lysosome-targeting structural unit, and/or the cell-penetrating peptide were fused to the C-terminus of 7D12 to prepare chimeras. However, the chimeras according to the invention are not limited to the amino acid sequences provided in this Example. The linker peptides Linker1 to Linker6, lysosome-targeting structural units P1 to P23, and cell-penetrating peptides C1 to C8 provided in the present disclosure can be combined in any manner. Furthermore, the lysosome-targeting structural units and cell-penetrating peptides can also be fused to the N-terminus of 7D12. All chimeras thus obtained fall within the scope of the invention.

The construction of plasmids for expressing 7D12-based chimeras in this Example specifically included the following steps.

First, a His₆ tag was fused to the C-terminus of 7D12 to construct fragment 7D12-His₆; and then the linker peptide Linker4 was fused with P1 and/or the cell-penetrating peptide C3 to the C-terminus of the His₆, to construct target fragments for expressing chimeras. These target fragments were respectively integrated into the pET-28a vector, and plasmids for expressing chimeras 7D12-P1 and 7D12-C3-P1 were obtained. The amino acid sequences of the plasmids for expressing chimeras 7D12-P1 and 7D12-C3-P1 are shown in Table 13.

TABLE 13
Amino acid sequences of chimeras

| Name | Target fragment | SEQ ID NO: |
|---|---|---|
| 7D12 | 7D12-His₆ | 131 |
| 7D12-P1 | 7D12-His₆-Linker4-P1 | 132 |
| 7D12-C3-P1 | 7D12-His₆-Linker4-C3-P1 | 133 |

### b) Expression, Purification and Characterization of Chimeras

The aforementioned plasmids for expressing chimeras were transformed into E. coli BL21 (DE3) host cells for protein expression, and the expressed products were purified using Ni-NTA beads.

The purification column was first equilibrated with an equilibration buffer (400 mM NaCl, 50 mM Tris-HCl, pH 8.0, 20 mM imidazole), and then the cell lysate supernatant was loaded onto the column. After loading, the column was washed with the same equilibration buffer to remove non-specifically bound proteins. Finally, the target protein was eluted using an elution buffer containing 200 mM imidazole. The eluted protein was concentrated and buffer-exchanged, and its concentration was determined using the BCA method.

### 3) Construction of Chimeras Based on the Polypeptides

### a) Design of the Polypeptides

In this Example, polypeptides PEP1 and PEP2 were used as parent polypeptides, which were structurally engineered to obtain polypeptides targeting lysosomes. P1 was used as the lysosome-targeting structural unit, and C3 or C4 was used as the cell-penetrating peptide. The lysosome-targeting structural unit and/or the cell-penetrating peptide were fused to the C-termini of the parent polypeptides to prepare chimeras based on the polypeptides, i.e., PEP1-P1, PEP1-C3-P1, and PEP2-C4-P1. The amino acid sequences of the chimeras PEP1-P1, PEP1-C3-P1, and PEP2-C4-P1 are shown in Table 14. However, the engineered polypeptides according to the invention are not limited to the amino acid sequences provided in this Example. The lysosome-targeting structural units P1 to P23, and cell-penetrating peptides C1 to C8 provided in the present disclosure can be combined in any manner, and all chimeras thus obtained fall within the scope of the invention.

TABLE 14
Amino acid sequences of chimeras

| Name | Target fragment | SEQ ID NO: |
|---|---|---|
| PEP1 | PEP1 | 36 |
| PEP1-P1 | PEP1-P1 | 134 |
| PEP1-C3-P1 | PEP1-C3-P1 | 135 |
| PEP2 | PEP2 | 37 |
| PEP2-C4-P1 | PEP2-C4-P1 | 136 |

### b) Expression, Purification and Characterization of Chimeras

The chimeras in this example were synthesized using the following steps.

First, Rink Amide MBHA resin (Novabiochem) was weighed and swollen in dichloromethane (DCM) for one hour, followed by thorough washing of the resin with N,N-dimethylformamide (DMF) three times. Using the Rink Amide MBHA resin as the solid support, a condensation reaction was performed with the coupling reagent (6-chloro-benzotriazole-1,1,3,3-tetramethyluronium hexafluorophosphate (HBTU) and the organic base N,N-diisopropylethylamine (DIEPA) in a 1:1 molar ratio), using DMF as the solvent, to sequentially couple the amino acids. After the final amino acid was coupled and deprotected, an appropriate amount of methanol was added to shrink the resin and remove residual DMF solvent. The resin was then treated with a cleavage cocktail of TFA/thioanisole/H₂O (95:2.5:2.5, v/v/v) for 2.5 hours. The mixture obtained then was filtered, and the filter cake was washed three times, and the filtrates were combined. The combined filtrate was slowly poured into ice-cold diethyl ether under stirring. The solution formed was allowed to stand for over 2 hours to ensure complete precipitation. The precipitate was collected by centrifugation and washed three times with ice-cold diethyl ether to obtain the crude compound. Finally, the crude product was purified and separated by semi-preparative liquid chromatography (column: Hypersil GOLD™ 5 μm C18, 21.2×250 mm) and the synthesized chimera was characterized by high-resolution mass spectrometry.

### Example 2: Chimera-Induced Degradation of HER2

Human Epidermal Growth Factor Receptor 2 (HER2) is a member of the epidermal growth factor receptor family. Its overexpression is associated with tumorigenesis, progression, enhanced invasiveness, and increased mortality, making it an important target in cancer therapy. In this Example, HER2 was selected as the protein of interest for degradation to validate whether the chimeras according to the invention can induce the degradation of HER2 on the surface of tumor cells. The ability of the chimeras to induce HER2 degradation was evaluated by detecting the expression level of HER2 on the cell membrane after treatment with the chimeras, using Western blot, flow cytometry, and confocal laser microscopy.

### 1) Expression Level of HER2 Detected by Western Blotting

In this Example, three HER2-high-expressing cell lines—SKBR3, SKOV3, and BT474—were used for the experiment. The cells were plated one day before the administration of chimeras. Each of the constructed chimeras was diluted in complete medium and incubated with one of the three cell lines for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes carrying the proteins were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-HER2 antibody.

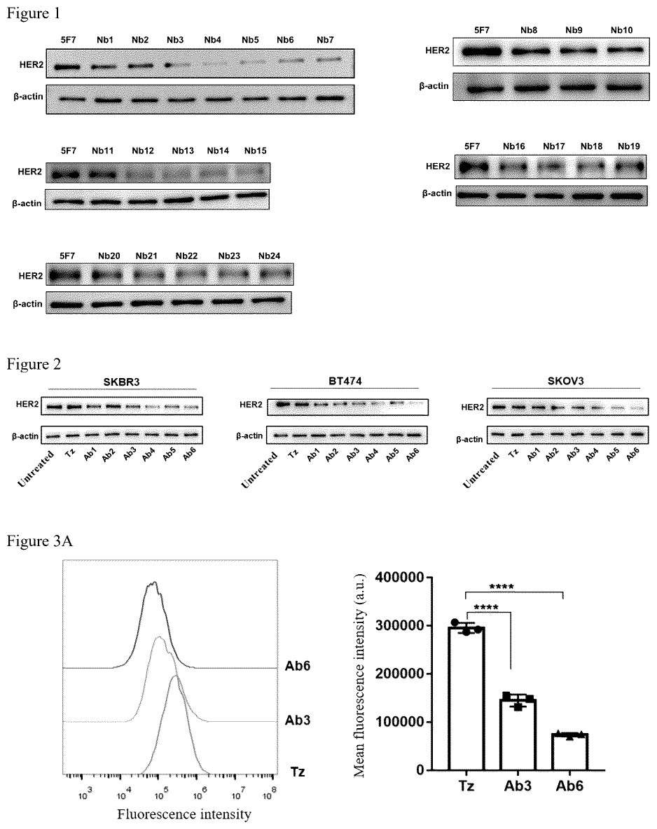

FIG. 1 displays Western blot images showing the degradation of the cell surface membrane protein HER2 via 24 different chimeras (Nb1 to Nb24) in this Example. The chimeras Nb1 to Nb24 were constructed by fusing the signal polypeptides P1, C3-P1, and P2 to P23 provided by the present disclosure to the nanobody 5F7. This experiment demonstrates that engineering antibodies using one of the lysosome-targeting structural units P1 to P23, either alone or in combination with a cell-penetrating peptide, can promote the degradation of the targets bound by the antibodies.

FIG. 2 displays Western blot images showing the degradation of the membrane protein HER2 on three different tumor cells (SKBR3 human breast adenocarcinoma cells, BT474 human breast cancer cells, and SKOV3 human ovarian cancer cells) via 6 different chimeras (Ab1 to Ab6 based) in this Example. The chimeras Ab1 to Ab6 were constructed by fusing the signal polypeptides P1 or C3-P1 provided by the present disclosure to the full-length antibody Tz at different positions. This experiment demonstrates that engineering antibodies in different manners using one of the lysosome-targeting structural units, alone or combined with a cell-penetrating peptide, can promote the degradation of targets bound by the antibodies.

As shown in the images, Ab1 to Ab6 induced significant degradation of HER2 in SKBR3, BT474, and SKOV3 cells, with significant differences compared to Tz. These results demonstrated that the chimeras incorporating signal sequences mediating the transport of the protein of interest to the lysosome induced obvious HER2 degradation. Moreover, the different fusions exhibited notable efficiency in degrading HER2 across different cell lines, indicating that all these construction manners can promote the degradation of the cell surface antigen HER2.

### 2) Level of Cell Membrane HER2 Detected by Flow Cytometry

In this Example, the cell line SKBR3 was used for the experiment. First, each of Tz, Ab3, and Ab6 was diluted in complete medium to a final concentration of 100 nM and then co-incubated with the SKBR3 cells for 48 hours. After incubation, the supernatant was discarded, and the cells were trypsinized and transferred to EP tubes. Then the cells were fixed in 4% paraformaldehyde at room temperature, followed by incubation with 1% BSA at room temperature for 1 hour. After the BSA treatment, the cells were first incubated with a primary antibody at 37° C. for 1 hour, and then incubated with a fluorescently labeled secondary antibody at room temperature for 30 minutes. The treated cells were observed for fluorescence using a flow cytometer, and the fluorescence intensity was analyzed with the FlowJo software.

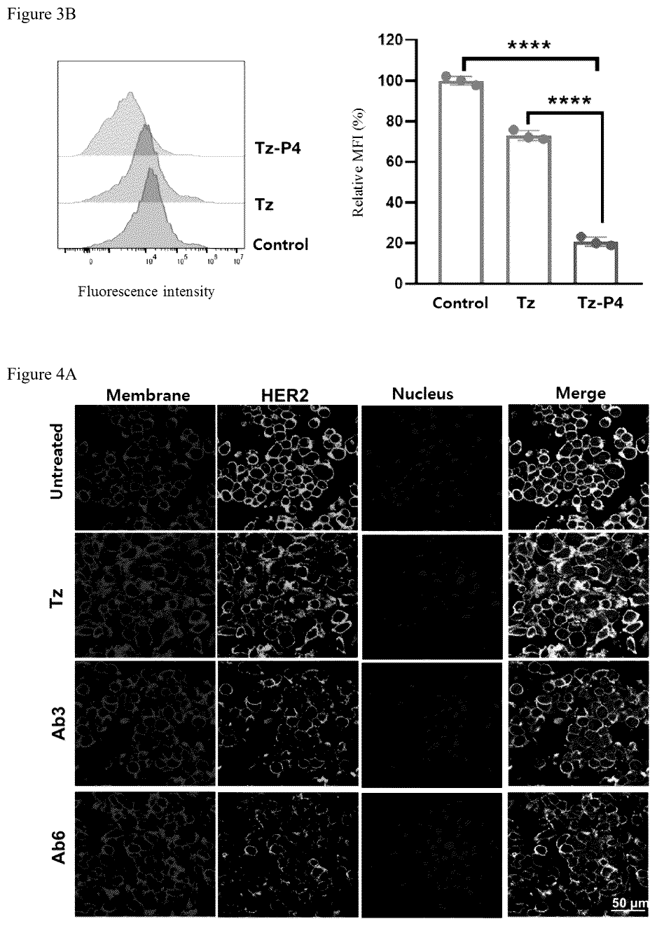

FIGS. 3A and 3B display plots showing the flow cytometry analysis and quantification results of the degradation of HER2 via Ab3, Ab6, and Tz-P4 in this Example. The chimeras Ab3, Ab6, and Tz-P4 were constructed by fusing the signal polypeptides P1, C3-P1, and P4 provided by the present disclosure to the light chain and the heavy chain of the full-length antibody Tz. Data are presented as the mean s.e.m. (n=3); **** P<0.0001. The Tz group in the plots represents the control group without the signal mediating the transport of the protein of interest to the lysosome fused. Flow cytometry analysis and the quantification of the mean cell fluorescence intensity clearly showed that the chimeras (Ab3, Ab6, and Tz-P4) with fusion engineering exhibited weaker fluorescence compared to Tz, indicating their enhanced ability to induce HER2 degradation. These results demonstrate that the chimeras fused with signals targeting the lysosome can obviously induce the degradation of HER2 on the cell membrane and the chimeras engineered using signals targeting the lysosome can promote the degradation of the target.

### 3) HER2 on the Cell Membrane Observed by Confocal Laser Microscopy

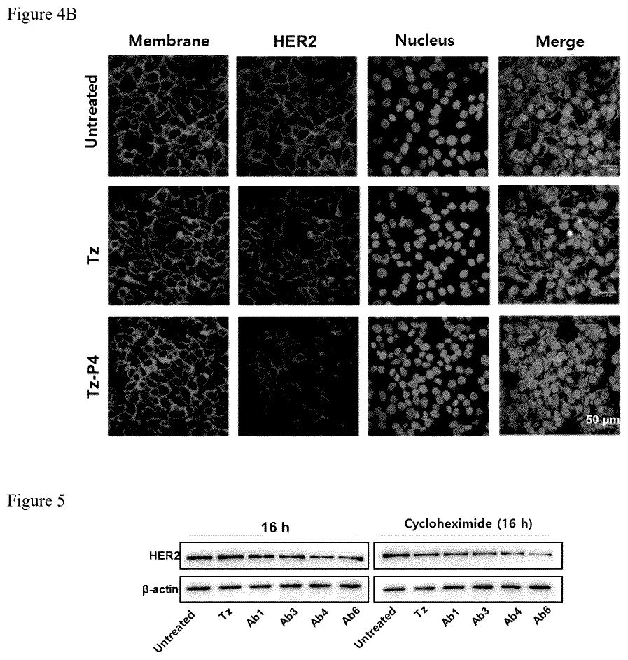

In this Example, the cell line SKBR3 was used for the experiment. First, each of Tz, Ab3, Ab6, and Tz-P4 was diluted in complete medium to a final concentration of 100 nM and then co-incubated with the SKBR3 cells. Next, the cell membrane was stained using cell membrane staining kit (purchased from Beyotime Biotechnology). After the staining, the cells were fixed in 4% paraformaldehyde at room temperature and incubated with 1% BSA at room temperature for 1 hour. Following the BSA treatment, the cells were first incubated with a primary antibody at 37° C. for 1 hour, then incubated with a fluorescently labeled secondary antibody at room temperature for 30 minutes. Nuclei were counterstained with DAPI. The expression of HER2 on the cell membrane after incubating the cells with Tz, Ab3, Ab6, and Tz-P4 was observed using a confocal laser scanning microscope, with results shown in FIG. 4.

FIGS. 4A and 4B display, from left to right, the signal from the cell membrane, the signal from HER2 on the cell membrane, the signal from the nuclei, and the merged signals of all three. The merge of the signals from HER2 on the cell membrane and from the cell membrane appears yellow. The signal from HER2 on the cell membrane showed that compared to Tz, the fluorescence intensity of HER2 on the surface of the cells which were treated with Ab3, Ab6, and Tz-P4 was lower, indicating a decrease in HER2 expression. This demonstrates that the chimeras can induce the degradation of HER2 on the cell membrane. The experiment further demonstrates that the chimeras engineered using the signals targeting the lysosome can promote the degradation of the target.

### 4) Inhibition of Protein Synthesis by Cycloheximide

In this Example, the cell line SKBR3 was used for the experiment. First, the cells were co-incubated with cycloheximide (50 μM) for 30 minutes. A mixture containing a chimera (at a concentration of 100 nM) and cycloheximide (at a concentration of 50 μM) was then prepared and co-incubated with the cells for 16 hours. Subsequently, the cells were lysed using a lysis buffer, total proteins were extracted, and protein concentration was quantified using a BCA protein assay kit. Proteins were separated by 10% SDS-PAGE and transferred onto polyvinylidene fluoride (PVDF) membranes via electroblotting. The membranes carrying the proteins were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ.

FIG. 5 displays Western blot images showing the HER2 levels after cycloheximide inhibition and incubation with Tz, Ab1, Ab3, Ab4, and Ab6 in this Example. Since cycloheximide is a bacterial toxin that interferes protein biosynthesis, its application blocks HER2 regeneration in the treated cells. Thus, the degradation of HER2 can be more clearly observed within a specific time frame. As shown in the images, the treatment of all the chimeras demonstrated enhanced degradation of HER2 following the addition of cycloheximide. These results demonstrate that the chimeras according to the invention can enhance HER2 degradation. Specifically, the data confirm that the chimeras according to the invention can accelerate the degradation of HER2 on the surface of tumor cells.

### Example 3: Chimera-Induced Degradation of EGFR

Epidermal Growth Factor Receptor (EGFR) is another key member of the epidermal growth factor receptor family and represents an important target in cancer therapy. In this Example, EGFR was selected as the protein of interest for degradation to validate whether the chimeras according to the invention can induce the degradation of various membrane proteins. This Example also involves the construction of chimeras based on full-length antibodies, nanobodies, and polypeptides, aiming to verify whether the degradation strategy according to the invention is applicable to chimeras of different molecular types and/or molecular weight ranges.

In this Example, two EGFR-high-expressing cell lines-A431, and A549-were selected for the experiment. The cells were plated one day before the administration of chimeras. Each of Ctx, Ctx-C3-P1, 7D12, 7D12-P1, 7D12-C3-P1, PEP1, PEP1-P1 and PEP1-C3-P1 was diluted in complete medium and incubated with the cells for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-EGFR antibody.

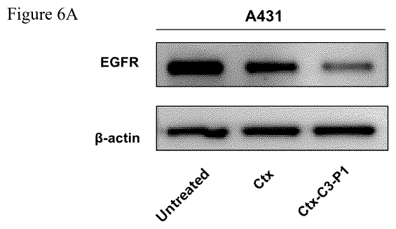

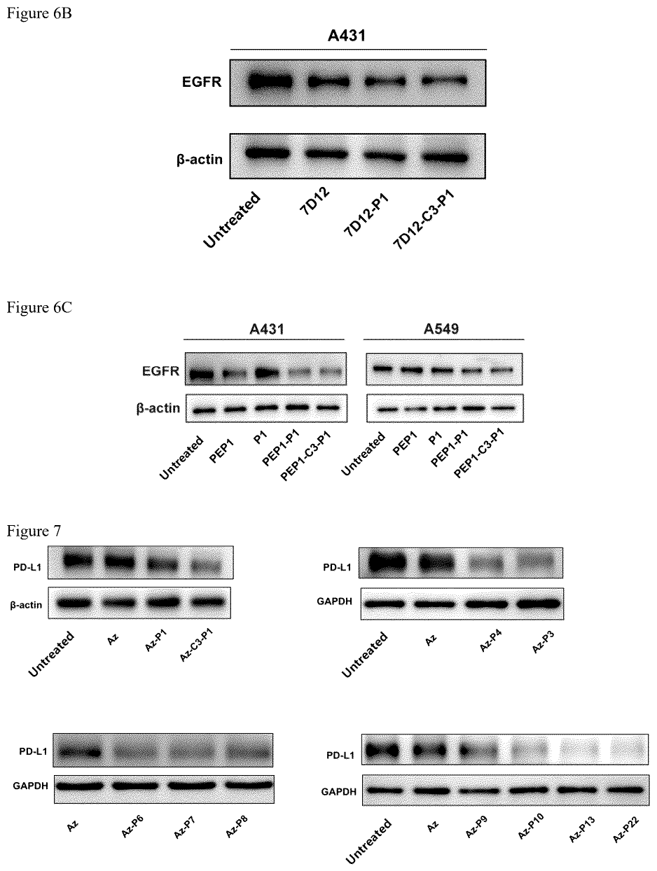

FIG. 6A displays Western blot images showing the degradation of EGFR via Ctx and Ctx-C3-P1 in this Example, with β-actin as the internal control and the “Untreated” representing the EGFR level in untreated cells. As shown in the images, Ctx-C3-P1 which fused with the lysosome-targeting structural unit exhibited a more pronounced degradation effect on EGFR compared to Ctx without signal fused. These results demonstrate that the full-length antibody-based chimera has enhanced ability to induce EGFR degradation, and that the chimera according to the invention is applicable to the degradation of different proteins of interest.

FIG. 6B displays Western blot images showing the degradation of EGFR via 7D12, 7D12-P1, and 7D12-C3-P1 in this Example, with β-actin as the internal control and the “Untreated” representing the EGFR level in untreated cells. As shown in the images, 7D12-P1 and 7D12-C3-P1 which fused with the lysosome-targeting structural unit exhibited a more pronounced degradation effect on EGFR compared to 7D12 without signal fused. These results demonstrate that the nanobody-based chimeras have enhanced ability to induce EGFR degradation, and that the chimeras according to the invention are applicable to the degradation of different proteins of interest.

FIG. 6C display Western blot images showing the degradation of EGFR via PEP1, PEP1-P1, and PEP1-C3-P1 in this Example, with β-actin as the internal control and the “Untreated” representing the EGFR level in untreated cells. In this Example, the peptides LARLLT and P1 were also used as controls. As shown in the images, both PEP1-P1 and PEP1-C3-P1 exhibited an obvious degradation effect on the membrane receptor EGFR in A431 and A549 cells, indicating that this construction strategy is also applicable with respect to polypeptide-based molecules.

This experiment demonstrates that the degradation strategy according to the invention is applicable to constructing chimeras of different molecular types and/or molecular weight ranges. Furthermore, the strategy according to the invention is suitable for degrading different membrane proteins.

### Example 4: Chimera-Induced Degradation of PD-L1

Programmed Cell Death Ligand 1 (PD-L1) was selected as the protein of interest. PD-L1 is an immune checkpoint molecule located on the cell surface, primarily functioning to regulate immune responses by preventing excessive immune reactions and autoimmune attacks. Overexpression of PD-L1 can lead to tumor immune evasion mechanisms. In this Example, PD-L1 was selected as the protein of interest for degradation to further validate whether the construction approach of chimera has a broad applicability across diverse targets.

### 1) Expression Level of PD-L1 Detected by Western Blotting

In this Example, the PD-L1-high-expressing cell line—MDA-MB-231 human breast cancer cells—was selected for the experiment. The cells were plated one day before the administration of chimeras. Each of Az, Az-P1, Az-C3-P1, Az-P3, Az-P4, Az-P6, Az-P7, Az-P8, Az-P9, Az-P10, Az-P13 and Az-P22 was diluted in complete medium and incubated with the cells for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-PD-L1 antibody.

FIG. 7 displays Western blot images showing the degradation of PD-L1 via 11 different chimeras (Az-P1, Az-C3-P1, Az-P3, Az-P4, Az-P6, Az-P7, Az-P8, Az-P9, Az-P10, Az-P13, and Az-P22) in this Example, with β-actin or GAPDH as the internal control and the “Untreated” representing the PD-L1 level in untreated cells. Further, Az was used as a control in this Example. The aforementioned 11 chimeras were constructed by fusing various lysosome-targeting structural units or a combination of a cell-penetrating peptide and the lysosome-targeting structural units provided by the present disclosure to the full-length antibody Az. As shown in the images, all the chimeras exhibited an obvious degradation effect on PD-L1 compared to the control Az, indicating they can induce the degradation of various types of proteins of interest including immune checkpoint molecules. The results demonstrate that this construction approach is applicable to various proteins of interest and the chimeras according to the invention have a universal applicability in degrading the proteins of interest bound by the chimeras.

### 2) PD-L1 on the Cell Membrane Observed by Confocal Laser Microscopy

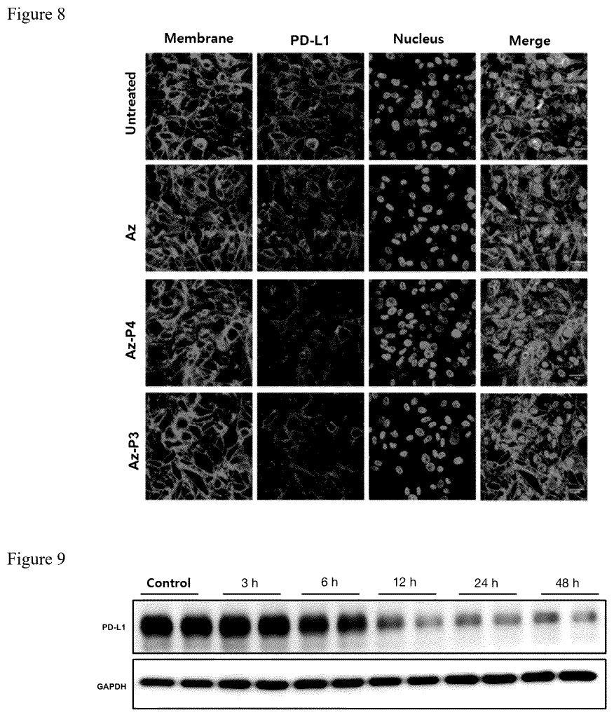

In this Example, the cell line MDA-MB-231 was used for the experiment. First, each of Az, Az-P3 and Az-P4 was diluted in complete medium to a final concentration of 100 nM and then co-incubated with the MDA-MB-231 cells. Next, the cell membrane was stained using a cell membrane staining kit (purchased from Beyotime Biotechnology). After the staining, the cells were fixed in 4% paraformaldehyde at room temperature and incubated with 1% BSA at room temperature for 1 hour. Following the BSA treatment, the cells were first incubated with a primary antibody at 37° C. for 1 hour, then incubated with a fluorescently labeled secondary antibody at room temperature for 30 minutes. Nuclei were counterstained with DAPI. The expression of PD-L1 on the cell membrane after incubating the cells with Az, Az-P3 and Az-P4 was observed using a confocal laser scanning microscope, with results shown in FIG. 8.

FIG. 8 displays confocal laser scanning microscopy images showing the degradation of PD-L1 on the MDA-MB-231 cells via two chimeras (Az-P3 and Az-P4) in this Example, and shows, from left to right, the signal from the cell membrane, the signal from PD-L1 on the cell membrane, the signal from the nuclei, and the merged signals of all three. The merge of the signals from PD-L1 on the cell membrane and from the cell membrane appears yellow. The signal from PD-L1 on the cell membrane showed that compared to Az, the fluorescence intensity of PD-L1 on the surface of cells which were treated with the chimeras was lower, indicating a decrease in PD-L1 expression. This demonstrates that the chimeras according to the invention can effectively achieve the degradation of PD-L1.

### 3) Time Course Study of PD-L1 Degradation

In this Example, the time course of PD-L1 degradation was examined by assessing the degradation of PD-L1 over time in MDA-MB-231 cells treated with Az-P4. The PD-L1 level was detected by Western blotting in this Example.

The PD-L1 degradation on MDA-MB-231 cells over time was evaluated in this experiment. FIG. 9 displays Western blot images showing the degradation of PD-L1 via the chimera at different time points in this Example. As shown in the images, the chimera achieved degradation of a substantial portion of PD-L1 after 12 hours. The data further demonstrate that the chimera according to the invention can efficiently achieve the degradation of target molecules.

### Example 5: Chimera-Induced Degradation of CD20

In this Example, another tumor-associated membrane protein, CD20, was selected as the protein of interest for degradation to investigate whether the chimera according to the invention can induce CD20 degradation. CD20 is an important tumor-specific antigen or molecular target. An additional reason for selecting CD20 as the protein of interest for degradation is that studies have shown it is a non-internalizable target, meaning it cannot internalize into cells on its own. This Example aims to investigate whether the chimera according to the invention can induce the internalization of non-internalizable targets into the lysosome and facilitate their degradation.

In this Example, the CD20-high-expressing cell line—Ramos B lymphocytic lymphoma cells—was selected for the experiment. The cells were plated one day before the administration of chimeras. Each of Rtx, Rtx-C3-P1, Oftm and Oftm-C3-P1 was diluted in complete medium and incubated with the cells for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes carrying the proteins were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-CD20 antibody.

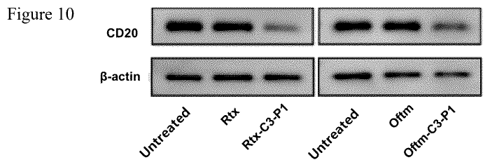

FIG. 10 displays Western blot images showing the degradation of CD20 via two chimeras (Rtx-C3-P1 and Oftm-C3-P1) in this Example. As shown in the images, the chimera Rtx-C3-P1 or Oftm-C3-P1 fused with a lyposome-targeting structural unit exhibited a more obvious degradation effect on CD20 compared to Rtx or Oftm without signal fused. CD20 is a non-internalizable target, and this experiment demonstrated that the chimera according to the invention can not only induce the degradation of internalizable targets (e.g., HER2, EGFR and PD-L1), but also induce the degradation of non-internalizable targets.

### Example 6: Chimera-Induced Degradation of CD71

In this Example, another tumor-associated membrane protein, CD71, was selected as the protein of interest for degradation to investigate whether the chimera according to the invention can induce CD71 degradation. CD71 is an important tumor-specific antigen or molecular target. Also known as transferrin receptor 1 (TfR1), CD71, after binding to iron-loaded transferrin, is rapidly internalized and recycled back to the cell surface instead of being delivered to lysosomes, thus evading degradation. This Example aims to investigate whether the chimera according to the invention can redirect the intracellular trafficking pathway of such recycling receptors to lysosomes, and further achieve their degradation.

In this Example, the CD71-high-expressing cell line—Jurkat human T lymphocyte leukemia cells—was selected for the experiment. The cells were plated one day before the administration of chimeras. Each of anti-CD71 and anti-CD71-C3-P1 was diluted in complete medium and incubated with the cells for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-CD71 antibody.

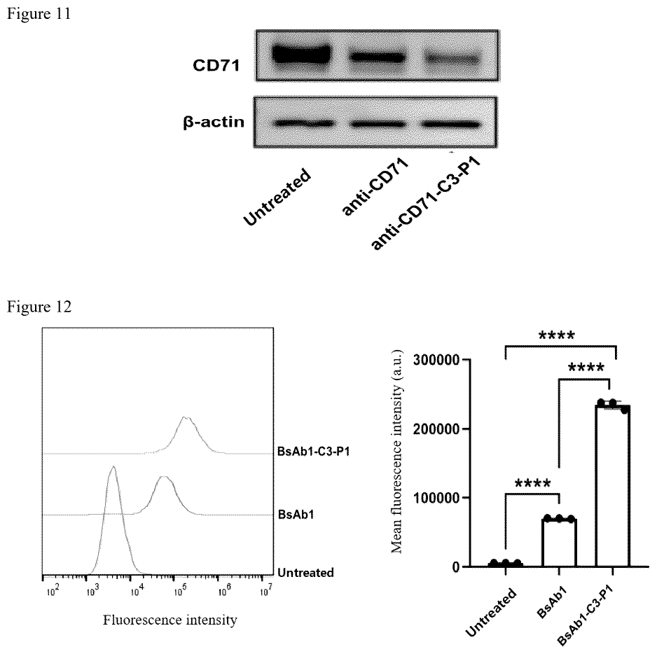

FIG. 11 displays Western blot images showing the degradation of CD71 via a full-length antibody-based chimera (anti-CD71-C3-P1) in this Example. As shown in the images, the chimera anti-CD71-C3-P1 fused with a lyposome-targeting structural unit exhibited a more obvious degradation effect on CD71 compared to anti-CD71 without signal fused. CD71 is a recycling receptor, and after binding to iron-loaded transferrin, it is rapidly internalized and recycled back to the cell surface instead of being delivered to lysosomes, thus evading degradation. The results demonstrate that the chimera according to the invention can redirect the intracellular trafficking pathway of such recycling receptors, and further achieve their degradation. The data further demonstrate that the chimera according to the invention can induce the degradation of membrane targets of various types.

### Example 7: Chimera-Induced Degradation of Extracellular Target VEGF

Vascular Endothelial Growth Factor (VEGF) is a ligand for cell surface receptors. It plays a critical role in tumor angiogenesis, and tumor growth and metastasis, and represents an important extracellular protein of interest. In this Example, VEGF was selected as the protein of interest to validate whether the chimera according to the invention can be applied to the degradation of extracellular targets.

### 1) Chimera-Induced Internalization of VEGF

In this Example, the cell line SKBR3 was first selected for the experiment. Fluorescent molecule FITC-labeled VEGF was diluted in serum-free medium to a final concentration of 50 nM, and then BsAb1 or BsAb1-C3-P1 at a final concentration of 100 nM was added to the diluted VEGF to prepare a mixed solution, which was then co-incubated with SKBR3 cells for 24 hours. Subsequently, an acid wash solution was used to remove antibodies bound to the cell membrane, and residual acid wash solution was rinsed off with PBS. The cells were then harvested from the culture dish and transferred to EP tubes. The treated cells were observed for fluorescence intensity using a flow cytometer.

FIG. 12 display plots showing the analysis and quantification results of the internalization of VEGF in this Example, in which the control group represents the background absorption of untreated cells. Data in FIG. 12 are presented as the mean±s.e.m. (n=3); **** P<0.0001. Flow cytometry analysis and the quantification of the mean cell fluorescence intensity clearly showed that compared to BsAb1, the chimera BsAb1-C3-P1 fused with a signal sequence mediating the transport of the protein of interest to the lysosome significantly facilitated the internalization of the extracellular protein VEGF. The results demonstrate that the chimera according to the invention can promote the internalization of extracellular proteins of interest.

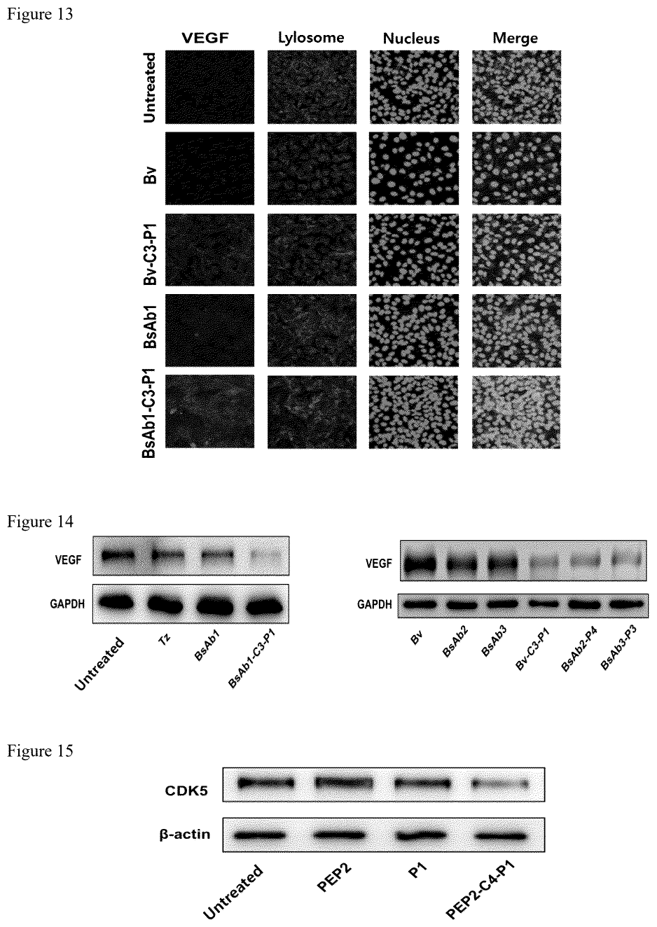

Subsequently, in this Example, the colocalization of VEGF with lysosomes was observed using a confocal laser scanning microscope. In this Example, the cell line SKBR3 was selected for the experiment. Each of Bv, Bv-C3-P1, BsAb and BsAb1-C3-P1 was diluted in complete medium to a final concentration of 100 nM and then co-incubated with the SKBR3 cells for 48 hours. Then the cells were fixed in 4% paraformaldehyde at room temperature, followed by incubation with 1% BSA at room temperature for 1 hour. After the BSA treatment, the cells were first incubated with a primary antibody at 37° C. for 1 hour, and then incubated with a fluorescently labeled secondary antibody at room temperature for 30 minutes. Nuclei were stained with 4′,6-diamidino-2-phenylindole (DAPI). The colocalization of FITC-labled VEGF with intracellular lysosomes was observed using a confocal laser scanning microscope, with results shown in FIG. 13.

FIG. 13 displays, from left to right, the signal from VEGF, the signal from lysosomes, the signal from the nuclei, and the merged signals of all three. The merge of the signals from lysosomes and VEGF appears orange. The “Untreated” cells in the Figure were used as the blank control group, and the cells treated with Bv or BsAb1 without signal sequences fused were used as negative control groups. The signal patterns of the chimeras in the Figure clearly showed that the green fluorescence in the Bv-C3-P1 and BsAb1-C3-P1 groups was obviously stronger than that in the Bv and BsAb1 groups, indicating that treatment with the chimeras promotes greater uptake of VEGF into the cells within the same incubation period. The results demonstrate that the chimera according to the invention can facilitate the entry of extracellular proteins into intracellular lysosomes.

### 2) Chimera-Induced Degradation of VEGF

In this example, the cell line SKBR3 was selected for the experiment. The cells were plated one day before the administration of chimeras. Each of BsAb1, BsAb1-C3-P1, Bv, BsAb2, BsAb3, Bv-C3-P1, BsAb2-P4 and BsAb3-P3 was diluted in complete medium and incubated with VEGF for 1 hour and then incubated with the cells for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes carrying the proteins were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-VEGF antibody.

FIG. 14 displays Western blot images showing the degradation of VEGF via the chimeras in this Example, with GAPDH as the internal control and the “Untreated” representing the VEGF level in untreated cells. In this Example, Tz, Bv, BsAb1, BsAb2, and BsAb3 were used as negative controls. As shown in the images, Bv-C3-P1, BsAb1-C3-P1, BsAb2-P4 and BsAb3-P3 exhibited a more obvious degradation effect on VEGF compared to the control groups. This experiment demonstrates that engineering antibodies in different manners using different signal peptides, alone or combined with a cell-penetrating peptide, can promote the degradation of extracellular targets bound by the antibodies. The data further demonstrate that the chimera according to the invention can efficiently achieve the efficient degradation of extracellular targets bound by the antibodies.

### Example 8: Chimera-Induced Degradation of Intracellular Target CDK5

Cycline-Dependent Kinase 5 (CDK5) is an important intracellular protein of interest. The overexpression of CDK5 is closely associated with tumorigenesis, progression, and metastasis. In this Example, CDK5 was selected as the protein of interest to validate whether the construction approach of chimera can be applied to the degradation of intracellular proteins of interest.

In this Example, a CDK5-high-expressing cell line—T24 human bladder cancer cells—was selected for the experiment. The cells were plated one day before the administration of chimeras. Each of PEP2, P1 and PEP-C4-P1 was diluted in complete medium and incubated with the cells for 48 hours. Subsequently, the cells were lysed using a lysis buffer, total proteins were extracted, and protein concentration was quantified using a BCA protein assay kit (Thermo Fisher). Proteins were separated by 10% SDS-PAGE and transferred onto polyvinylidene fluoride (PVDF) membranes via electroblotting. The membranes carrying the proteins were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ. All primary antibodies used in this Example were an anti-CDK5 antibody.

FIG. 15 displays Western blot images showing the degradation of CDK5 via PEP2, P1, and PEP2-C4-P1 in this Example, with β-actin as the internal control and the “Untreated” representing the CDK5 level in untreated cells. In this Example, the polypeptides PEP2 and P1 were also used as controls. As shown in the images, PEP2-C4-P1 exhibited an obvious degradation effect on CDK5, compared to PEP2 and P1. The results demonstrate that the chimera according to the invention can induce the degradation of intracellular proteins of interest.

### Example 9: Study on the Mechanism of Induced Protein Degradation

In this Example, the specific mechanism of protein degradation was primarily investigated through Western blotting, confocal laser microscopy, and electron microscopy. In this Example lysosome inhibitors leupeptin and chloroquine were used to observe whether HER2 degradation still occurred upon inhibition of lysosomal function. The experiment was conducted using the cell line SKBR3. First, the cells were co-incubated with leupeptin (0.1 mg/mL) or chloroquine (50 μM) for 30 minutes. Subsequently, mixtures of the chimeras and the lysosome inhibitors (with each of the chimeras at a concentration of 100 nM) were prepared and co-incubated with the cells for 24 hours. After incubation, the cells were lysed using a lysis buffer, total proteins were extracted, and protein concentration was quantified using a BCA protein assay kit. Proteins were separated by 10% SDS-PAGE and transferred onto polyvinylidene fluoride (PVDF) membranes via electroblotting. The membranes carrying the proteins were blocked with 5% skim milk at room temperature for 1 hour. After removing the milk, the membranes were incubated with a primary antibody overnight at 4° C. on a shaker. The next day, the membranes were quickly immersed in TBST and washed three times, followed by incubation with a secondary antibody at room temperature for 1 hour. Finally, the proteins were detected and recorded using a chemiluminescence reagent and a Tanon chemiluminescence imaging system, and quantitative analysis was performed on the proteins using ImageJ.

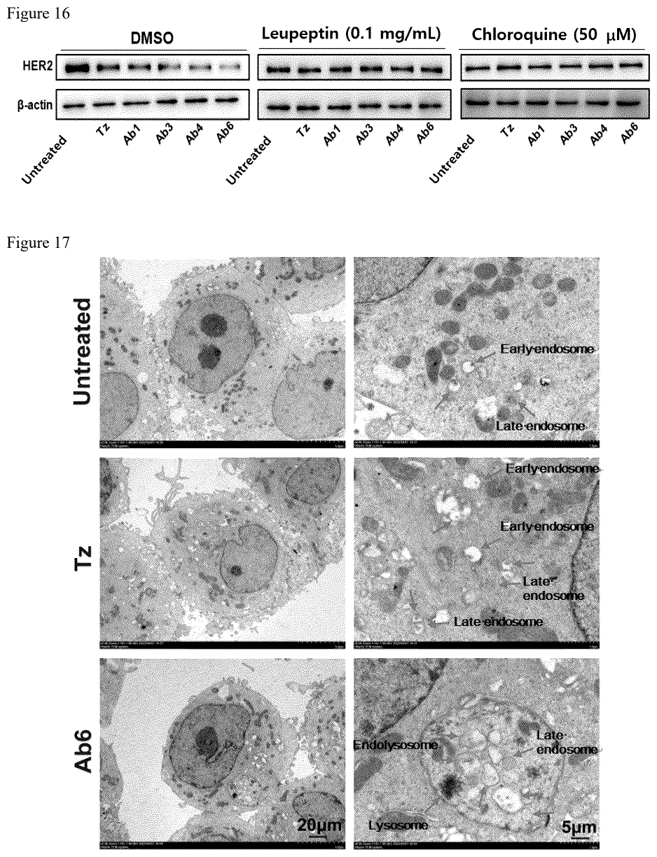

FIG. 16 displays Western blot images showing the HER2 levels after co-incubation with Tz, Ab1, Ab3, Ab4, Ab6 and lysosome inhibitors in this Example. As shown in the images, when the lysosomal function was not inhibited, the chimeras induced the degradation of HER2 on the cell membrane, with Ab1, Ab3, Ab4, and Ab6 showing significant degradation effects compared to Tz. In contrast, when the lysosomal function was inhibited, neither Tz nor Ab1, Ab3, Ab4, and Ab6 induced degradation of HER2 on the cell surface, with consistent results observed for both inhibitors. The results demonstrate that HER2 degradation occurs via the endocytosis-lysosome pathway, meaning the chimeras according to the invention achieve the degradation by directing target molecules into lysosomes.

In this Example, an electron microscope was further used to observe the changes in intracellular lysosomes and lysosome-related organelles including early endosomes, late endosomes, and endolysosomes. FIG. 17 displays electron microscopy images showing lysosomes and lysosome-related organelles in cells after treatment with Tz and Ab6 respectively in this Example, with the “Untreated” representing the electron microscopy images of untreated normal cells. In the normal cells, early endosomes, late endosomes, and lysosomes were observed; in the Tz group, an obvious increased number of early endosomes, late endosomes, and lysosomes was seen; in the Ab6 group, not only were early endosomes, late endosomes, endolysosomes, and lysosomes more abundant compared to the Tz group, but the lysosomes were also larger and more numerous. The data demonstrated that the administration of the chimeras led to an increased number of lysosomes, thereby promoting the degradation of the target. The results further demonstrate the crucial role of lysosomes in HER2 degradation and indicate that the chimeras according to the invention enhance the efficiency of HER2 degradation by promoting the proliferation of lysosomes.

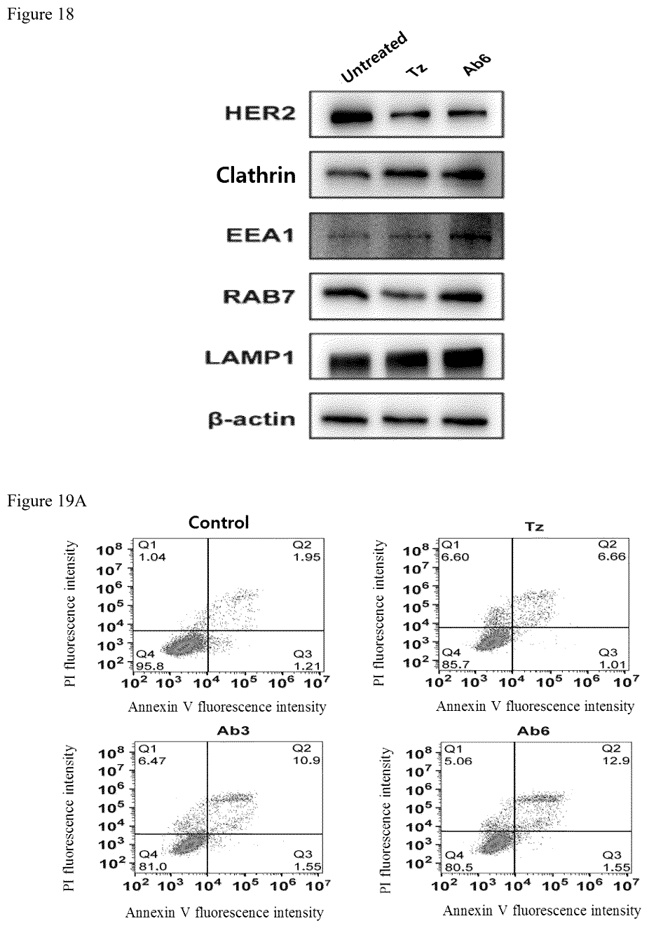

Additionally, in this Example the pathway by which Ab6 induces the degradation of protein of interest was investigated by detecting key proteins in the endocytosis-lysosome pathway via Western blotting. FIG. 18 displays Western blot images showing the levels of protein markers associated with the endocytosis-lysosome pathway after administration of Tz and Ab6 respectively in this Example. The “Untreated” represents the blank control groups, and Tz was used as a negative control. As shown in the images, the levels of the protein markers EEA1 (early endosome), RAB7 (late endosome) and LAMP1 (lysosome) were significantly increased after the cells were incubated with Ab6. These data further demonstrate that the lysosome-targeting signals disclosed by the present disclosure achieve the degradation of target molecules through the endocytosis-lysosome pathway.

In summary, the above experimental results demonstrate that the chimera Ab6 provided in the present disclosure achieves degradation of the protein of interest HER2 via the endocytosis-lysosome pathway.

### Example 10: In Vitro Anti-Tumor Activity

### 1) Influence of Chimeras on Apoptosis

In this Example, SKBR3 cells were selected for detection. When cell density reached 60-70%, the cells were incubated in complete medium containing the chimeras for 48 hours. Then the cells were trypsinized and harvested, and the harvested cells were diluted in a buffer into a concentration of 10⁶ cells/mL. Next, 5 μL of Annexin V-FITC was added to each tube containing the cells, followed by incubation at room temperature for 10 minutes. 5 minutes before analysis by flow cytometry, 5 μL of PI staining solution was added. Finally, apoptosis of the cells was analyzed using a flow cytometer.

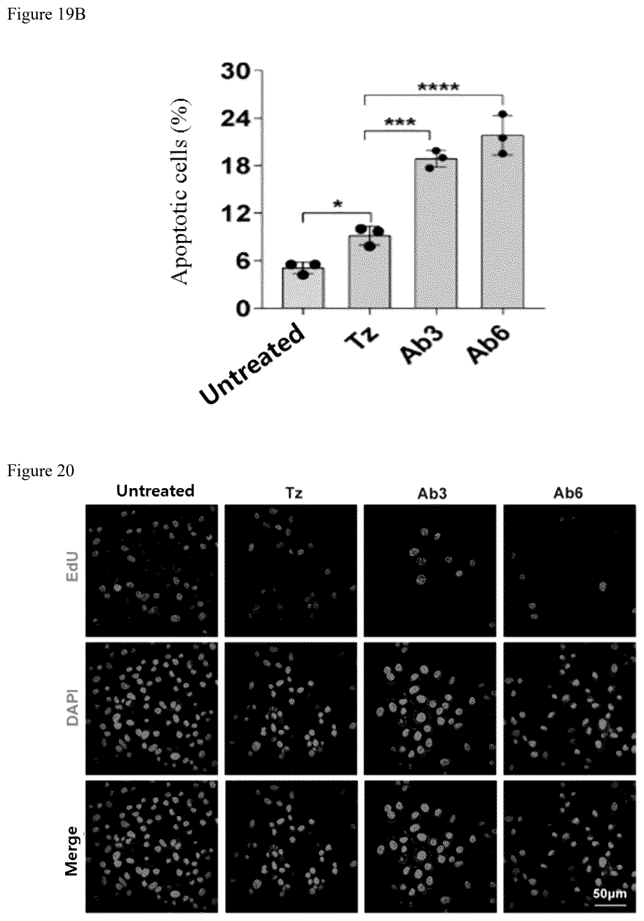

FIG. 19A displays plots showing the flow cytometry analysis results of apoptosis via Tz, Ab3, and Ab6 in this Example, and FIG. 19B displays a plot showing the quantification results of apoptosis via Tz, Ab3, and Ab6 in this Example. Data in the Figures are presented as the mean±s.e.m. (n=3); * P<0.05, *** P<0.001, and **** P<0.0001. The control group (Control) represents the background absorption of the cells. As shown in the plots, treatment with Ab3 and Ab6 yielded an increased percentage of apoptotic cells compared to Tz. The results demonstrate that the chimeras according to the invention can promote tumor cell apoptosis, thereby kill tumor cells, and thus possess in vitro anti-tumor activity.

### 2) Influence of Chimeras on Cell Proliferation

The assessment of cell proliferation capacity is a fundamental method for evaluating cell viability, genotoxicity, and the efficacy of anti-tumor drugs. 5-ethynyl-2′-deoxyuridine (EdU) is a novel thymidine analogue that can be incorporated into newly synthesized DNA during DNA synthesis, replacing thymidine.

In this Example, SKBR3 cells were used. Each of Tz, Ab3, and Ab6 was diluted in complete medium to a final concentration of 100 nM and then co-incubated with the SKBR3 cells. A 2×EdU working solution (purchased from Beyotime Biotechnology) was prepared (the final concentration was 10 μM (1×)). The 2×EdU working solution (20 μM) was pre-warmed at 37° C. and then added in a volume equal to the medium into the 6-well plate, resulting in a final EdU concentration of 1× in the plate. The cells were incubated for an additional 2 hours. After EdU labeling was complete, the culture broth was removed, and 1 mL of 4% paraformaldehyde was added to fix the cells at room temperature for 15 minutes. The fixing solution was removed, and the cells were washed three times with 1 mL of washing buffer per well, for 3 minutes each time. After removing the washing buffer, the cells were permeabilized with 1 mL of PBS containing 0.3% Triton X-100 per well and incubated at room temperature for 10 minutes. The permeabilization solution was removed, and the cells were washed once with 1 mL of a washing buffer per well for 3 minutes. A Click reaction solution was prepared, and 0.5 mL of this solution was added to each well, followed by incubation at room temperature in dark for 30 minutes. The cells were then washed three times with a washing buffer, for 3 minutes each time. Nuclei were stained with DAPI for 15 minutes, and EdU staining was observed under a fluorescence microscope. The image scaling and acquisition conditions were identical for all comparable images.

FIG. 20 displays confocal laser scanning microscopy images for Tz, Ab3, and Ab6 in this Example. From top to bottom, the images show the signal from EdU, the signal from the nuclei, and the merge of the signals of both. A stronger EdU signal indicates a larger number of normal cells. As shown in the images, Ab3 and Ab6 resulted in weaker EdU signals compared to Tz. The results demonstrate that the chimeras according to the invention fusing with the signal sequence mediating the transport of protein of interest to the lysosome, can significantly inhibit the proliferation of tumor cells, and thus possess in vitro anti-tumor activity.

### Example 11: In Vivo Anti-Tumor Activity

In this Example 50 SPF-grade nude/nude female mice were used as the experimental animals. The mice were purchased from Beijing Vital River Laboratory Animal Technology Co., Ltd. (Animal Use License No.: SCXK (Jing) 2021-0006), were 6-8 weeks old and weighed 20-25 g. All the mice were housed under a 12-hour light/dark cycle with unrestricted access to food and water, at room temperature and 55% humidity.

Grouping and Modeling: After one week of acclimatization, the mice were randomly divided into the following groups: a blank control group (n=5), and model, Tz, and Ab6 groups (each n=6). A suspension of SKOV3 cells (1×10⁷ cells) were subcutaneously inoculated into the right hind limb of each nude mouse with an inoculation volume of 0.2 mL. The model was considered successfully established when the tumor volume reached 100-150 mm³.

Administration Method: According to the grouping, each mouse was administered 15 mg/kg via intraperitoneal injection every 4 days. The model group received PBS injections.

Sample Collection and Detection: 24 hours after the last administration, the mice were euthanized by cervical dislocation. Subcutaneous tumor tissues were collected. The long and short diameters of the tumor tissues from the mice were measured and the tumor mass was weighed. Tumor volume was calculated using the formula: Tumor Volume=(Length×Width²)/2.

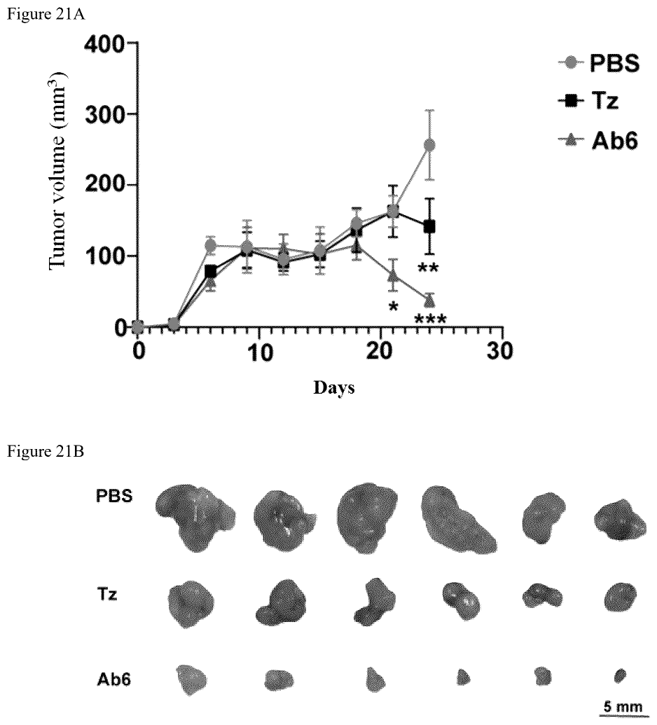

FIG. 21A displays a graph showing the tumor volume statistics of mice post-modeling in this Example. The administration commenced when the tumor volume reached 100 mm³. As shown in the graph, tumors in the model group continued to grow; the administration in the Tz group showed a certain inhibitory effect on tumor growth, while the administration in the Ab6 group showed obviously inhibited tumor growth, with a notable reduction in tumor tissue. The results demonstrate that the chimeras according to the invention can significantly inhibit tumor growth.

FIG. 21B displays photographs showing excised tumor tissues from mice in this Example. As shown in the photographs, Ab6 significantly inhibited tumor growth; furthermore, Ab6 exhibited a more pronounced inhibitory effect on tumor growth compared to the Tz group, consistent with the results above. The results demonstrate that the chimeras according to the invention can inhibit tumor cell growth and induce tumor regression.

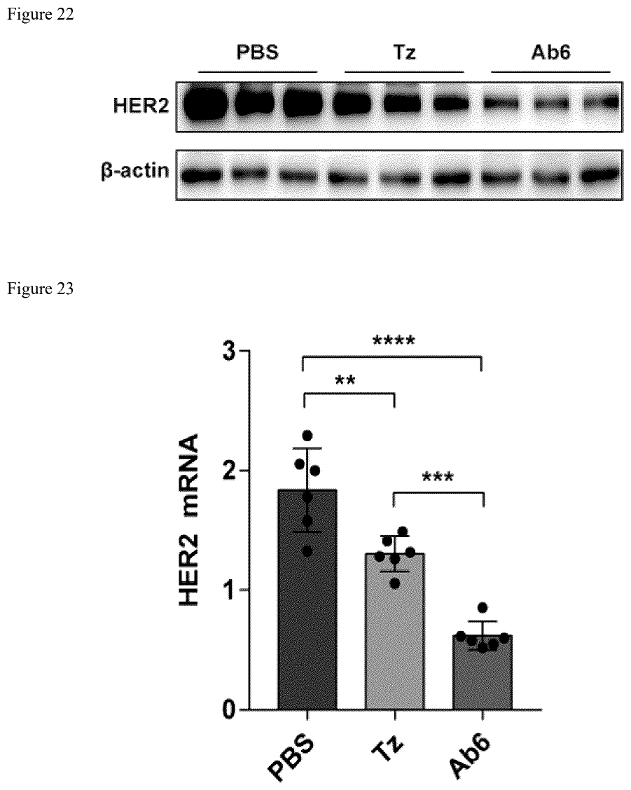

FIG. 22 displays Western blot images showing the HER2 levels in excised tumor tissues from mice in this Example. As shown in the images, HER2 expression levels in the tumor tissues from the mice of the Tz and Ab6 groups were lower than that in the model group; furthermore, the HER2 expression level in the tumor tissue from the mice of the Ab6 group was obviously lower than that in the Tz group. The results demonstrate that the chimeras according to the invention can induce the targeted degradation of HER2 on the surface of tumor cells and induce cell apoptosis, thereby inhibiting tumor growth.

FIG. 23 displays a plot showing the QPCR statistics of excised tumor tissues from mice in this Example. Proved at the mRNA level, Ab6 indeed reduced the content of HER2 mRNA compared to Tz. This is consistent with the Western blot results above, indicating that the chimera according to the invention can induce the targeted degradation of HER2 on the surface of tumor cells, thereby inhibiting tumor growth. The data further demonstrate that the chimera according to the invention can induce the targeted degradation of proteins of interest in tumor cells in vivo.

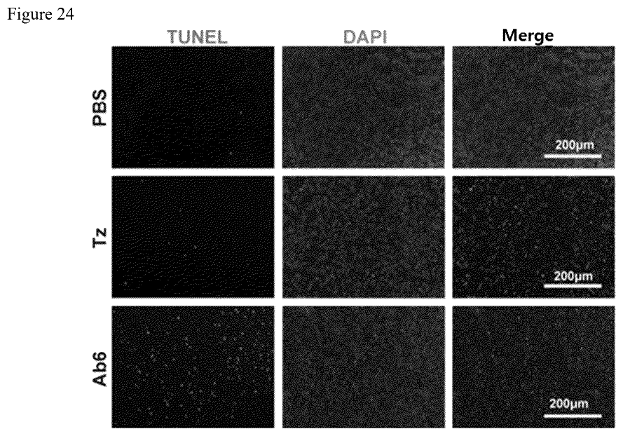

FIG. 24 displays images showing the TUNEL staining of excised tumor tissues from mice in this Example. As shown in the images, the apoptotic signal in the tumor tissues from the mice in the Ab6 group was significantly stronger than that in the Tz group. The results demonstrate that the chimera according to the invention can inhibit tumor cell growth by inducing tumor cell apoptosis.

According to the foregoing Examples, the chimera according to the invention can effectively achieve the enrichment and efficient degradation of various types of proteins of interest-located on the cell surface, extracellularly, and intracellularly-within lysosomes. Mechanism study provided in the present disclosure indicated that the chimeras according to the invention utilized lysosome-targeting structural unit to facilitate the degradation of proteins of interest in lysosomes via the endocytosis-lysosome pathway. A chimera fused with a lysosome-targeting signal sequence more effectively inhibited tumor cell proliferation and promote tumor cell apoptosis. Experiments in mice demonstrated that the chimeras according to the invention significantly reduced tumor size and growth rate. Collectively, these results indicate that the signal-mediated degradation technology provided in the present disclosure represents an effective strategy for disease treatment. The present disclosure provides the application of the chimera in targeted protein degradation technology, which can achieve efficient degradation of proteins of interest by constructing a signal-mediated, lysosome-targeting chimera.

The invention has been disclosed above with the embodiments, which however are not intended to limit the invention. Any person skilled in the art may make various modifications and refinements without departing from the spirit and scope of the invention. Therefore, the protection scope of the invention shall be defined by the scope of the appended claims.

1.-18. (canceled)

19. A method for targeted protein degradation, characterized in that the method comprises administering to a subject an effective amount of a chimera, the chimera comprising:
(1) a molecule selectively binding to a protein of interest; and
(2) a signal sequence mediating the transport of the protein of interest to the lysosome, which is directly or indirectly fused to the molecule.

20. The method according to claim 19, characterized in that the signal sequence mediating the transport of the protein of interest to the lysosome comprises at least one lysosome-targeting structural unit, or a combination of the at least one lysosome-targeting structural unit and at least one cell-penetrating peptide.

21. The use according to claim 20, characterized in that the lysosome-targeting structural unit is at least one selected from the group consisting of a lysosome-sorting signal peptide derived from Golgi-localized, gamma-adaptin ear-containing, Auxin response factor-binding (GGA) protein, a lysosome-sorting signal peptide derived from mannose-6-phosphate receptor (MPR), a lysosome-sorting signal peptide derived from lysosome-associated membrane protein (LAMP), and a mutant thereof, wherein the mutant is one of the lysosome-sorting signal peptide mutants of the lysosome-sorting signal peptide derived from GGA protein, the lysosome-sorting signal peptide derived from MPR, or the lysosome-sorting signal peptide derived from LAMP formed by the substitution, deletion, and/or addition of one or more amino acids.

22. The method according to claim 21, characterized in that the lysosome-targeting structural unit is at least one selected from the group consisting of the following amino acid sequences: SFHDDSDEDLLHI (SEQ ID NO: 1), DDSDEDLL (SEQ ID NO: 2), EESEERDDHLLPM (SEQ ID NO: 3), SYKYSKVNKE (SEQ ID NO: 4), YKYSKV (SEQ ID NO: 5), PAAYRGVGDD (SEQ ID NO: 6), RKRSHAGYQTI (SEQ ID NO: 7), ASSGLDDLDLLGK (SEQ ID NO: 8), VQNPSADRNLLDL (SEQ ID NO: 9), ASVSLLDDELMSL (SEQ ID NO: 10), RRRASVSLLDDELMSL (SEQ ID NO: 11), ASVSLLDDEL (SEQ ID NO: 12), NALSWLDEELLCL (SEQ ID NO: 13), SDEDLLHI (SEQ ID NO: 14), RRRSDEDLLHI (SEQ ID NO: 15), RRLRKSDEDLLHI (SEQ ID NO: 16), RRRRKSDEDLLHI (SEQ ID NO: 17), RRRSFHDDSDEDLLHI (SEQ ID NO: 18), RRLRKSFHDDSDEDLLHI (SEQ ID NO: 19), RRRRKSFHDDSDEDLLHI (SEQ ID NO: 20), RRRRKRKRSHAGYQTI (SEQ ID NO: 21), KHHHAGYEQF (SEQ ID NO: 22), RRLRKHHHAGYEQF (SEQ ID NO: 23), and a mutated amino acid sequence of any one of the amino acid sequences of SEQ ID NOs: 1-23 formed by the substitution, deletion, and/or addition of one or more amino acids,
preferably, the lysosome-targeting structural unit is at least one selected from the group consisting of SEQ ID NOs: 1-23.

23. The method according to claim 20, characterized in that the cell-penetrating peptide is at least one selected from the group consisting of the following amino acid sequences: RRR, RRLRK (SEQ ID NO: 24), RRRRK (SEQ ID NO: 25), YGRKKRRQRRR (SEQ ID NO: 26), LLIILRRRIRKQAHAHSK (SEQ ID NO: 27), RQIKIWFQNRRMKWKK (SEQ ID NO: 28), RRRRRRRRR (SEQ ID NO: 29) and GWTLNSAGYLLGKINLKALAALAKKIL (SEQ ID NO: 30).

24. The method according to claim 19, characterized in that the signal sequence mediating the transport of the protein of interest to the lysosome is indirectly fused to the molecule selectively binding to the protein of interest via a first linker peptide or a first chemical linker.

25. The method according to claim 20, characterized in that the lysosome-targeting structural unit is directly fused to the cell-penetrating peptide, or fused to the cell-penetrating peptide via a second linker peptide or a second chemical linker.

26. The method according to claim 24, characterized in that the amino acid sequence of the first or second linker peptide is independently (Leu-Pro-Glu-Thr)ₓ-(Gly_{y1}-Ser_{y2})_{z}, in which x=0 or 1, y1=3, 4, or 5, y2=0 or 1, and z=1, 2, or 3.

27. The method according to claim 26, characterized in that the first or second linker peptide is at least one independently selected from the group consisting of the following amino acid sequences: Leu-Pro-Glu-Thr-Gly-Gly-Gly (SEQ ID NO: 31), Gly-Gly-Gly, Gly-Gly-Gly-Gly-Gly (SEQ ID NO: 32), Gly-Gly-Gly-Gly-Ser (SEQ ID NO: 33), Gly-Gly-Gly-Gly-Ser-Gly-Gly-Gly-Gly-Ser (SEQ ID NO: 34), and Gly-Gly-Gly-Gly-Ser-Gly-Gly-Gly-Gly-Ser-Gly-Gly-Gly-Gly-Ser (SEQ ID NO: 35).

28. The method according to claim 19, characterized in that the molecule selectively binding to the protein of interest comprises a polypeptide, a protein, a nucleic acid, a nanoparticle, or a small molecule compound,
preferably, the polypeptide is glycosylated or non-glycosylated, or consists of natural amino acids and/or unnatural amino acids.

29. The method according to claim 28, characterized in that the signal sequence mediating the transport of the protein of interest to the lysosome is fused to the polypeptide at at least one of the C-terminus of the polypeptide, the N-terminus of the polypeptide, and an amino acid side chain group of the polypeptide;
preferably, the amino acid side chain group comprises a side chain group of a natural amino acid or a side chain group of an unnatural amino acid;
preferably, the side chain group of a natural amino acid comprises an amino group and a thiol group; and/or the side chain group of an unnatural amino acid comprises an azido group, alkyne group, aldehyde group, ketone group, fluoro sulfonate, chloro group, bromo group, or iodo group.

30. The method according to claim 28, characterized in that the protein is an antibody;
preferably, the antibody comprises a polyclonal antibody, a monoclonal antibody, a monospecific antibody, a bispecific antibody, a multispecific antibody, a chimeric antibody, a humanized antibody, a human antibody, or an antigen-binding fragment;
preferably, the antigen-binding fragment is a Fab, a Fab′, a F(ab′)₂, a Fv, a dsFv, a scFv, a sc(Fv)₂, or a VHH.

31. The method according to claim 30, characterized in that the signal sequence mediating the transport of the protein of interest to the lysosome is fused to the protein at at least one of the C-terminus of the heavy chain of the antibody, the N-terminus of the heavy chain of the antibody, the C-terminus of the light chain of the antibody, the N-terminus of the light chain of the antibody, an amino acid side chain group of the antibody, the C-terminus of the antigen-binding fragment, the N-terminus of the antigen-binding fragment, and an amino acid side chain group of the antigen-binding fragment.

32. The method according to claim 19, characterized in that the signal sequence mediating the transport of the protein of interest to the lysosome is fused to the molecule selectively binding to the protein of interest by a method comprising at least one of chemical conjugation, enzymatic catalysis, and genetic recombination.

33. The method according to claim 19, characterized in that the protein of interest is at least one of a cell surface protein, an intracellular protein, and an extracellular protein.

34. The method according to claim 19, characterized in that the protein of interest is at least one of a disease-associated protein of interest and an immune checkpoint molecule.

35. The method according to claim 34, characterized in that the disease comprises at least one of a cancer, an inflammation-related disease, an immune-related disease, a viral infection, a metabolic disease, and a neurodegenerative disease.

36. The method according to any claim 19, characterized in that the protein of interest is at least one of an internalizable or non-internalizable receptor, a ligand of a receptor, a cytokine, a hormone, a secreted protein, an antibody, a protein carrier, and an enzyme.

37. A chimera, comprising:
(1) a molecule selectively binding to a protein of interest; and
(2) a signal sequence mediating the transport of the protein of interest to the lysosome, which is directly or indirectly fused to the molecule.

38. The chimera according to claim 37, characterized in that the signal sequence mediating the transport of the protein of interest to the lysosome comprises at least one lysosome-targeting structural unit, or a combination of the at least one lysosome-targeting structural unit and at least one cell-penetrating peptide.

39. The chimera according to claim 38, characterized in that the lysosome-targeting structural unit is at least one selected from the group consisting of a lysosome-sorting signal peptide derived from Golgi-localized, gamma-adaptin ear-containing, Auxin response factor-binding (GGA) protein, a lysosome-sorting signal peptide derived from mannose-6-phosphate receptor (MPR), a lysosome-sorting signal peptide derived from lysosome-associated membrane protein (LAMP), and a mutant thereof, wherein the mutant is one of the lysosome-sorting signal peptide mutants of the lysosome-sorting signal peptide derived from GGA protein, the lysosome-sorting signal peptide derived from MPR, or the lysosome-sorting signal peptide derived from LAMP formed by the substitution, deletion, and/or addition of one or more amino acids.

40. The chimera according to claim 39, characterized in that the lysosome-targeting structural unit is at least one selected from the group consisting of the following amino acid sequences: SFHDDSDEDLLHI (SEQ ID NO: 1), DDSDEDLL (SEQ ID NO: 2), EESEERDDHLLPM (SEQ ID NO: 3), SYKYSKVNKE (SEQ ID NO: 4), YKYSKV (SEQ ID NO: 5), PAAYRGVGDD (SEQ ID NO: 6), RKRSHAGYQTI (SEQ ID NO: 7), ASSGLDDLDLLGK (SEQ ID NO: 8), VQNPSADRNLLDL (SEQ ID NO: 9), ASVSLLDDELMSL (SEQ ID NO: 10), RRRASVSLLDDELMSL (SEQ ID NO: 11), ASVSLLDDEL (SEQ ID NO: 12), NALSWLDEELLCL (SEQ ID NO: 13), SDEDLLHI (SEQ ID NO: 14), RRRSDEDLLHI (SEQ ID NO: 15), RRLRKSDEDLLHI (SEQ ID NO: 16), RRRRKSDEDLLHI (SEQ ID NO: 17), RRRSFHDDSDEDLLHI (SEQ ID NO: 18), RRLRKSFHDDSDEDLLHI (SEQ ID NO: 19), RRRRKSFHDDSDEDLLHI (SEQ ID NO: 20), RRRRKRKRSHAGYQTI (SEQ ID NO: 21), KHHHAGYEQF (SEQ ID NO: 22), RRLRKHHHAGYEQF (SEQ ID NO: 23), and a mutated amino acid sequence of any one of the amino acid sequences of SEQ ID NOs: 1-23 formed by the substitution, deletion, and/or addition of one or more amino acids,
preferably, the lysosome-targeting structural unit is at least one selected from the group consisting of SEQ ID NOs: 1-23.

41. The chimera according to claim 38, characterized in that the cell-penetrating peptide is at least one selected from the group consisting of the following amino acid sequences: RRR, RRLRK (SEQ ID NO: 24), RRRRK (SEQ ID NO: 25), YGRKKRRQRRR (SEQ ID NO: 26), LLIILRRRIRKQAHAHSK (SEQ ID NO: 27), RQIKIWFQNRRMKWKK (SEQ ID NO: 28), RRRRRRRRR (SEQ ID NO: 29) and GWTLNSAGYLLGKINLKALAALAKKIL (SEQ ID NO: 30).

42. The chimera according to claim 37, characterized in that the signal sequence mediating the transport of the protein of interest to the lysosome is indirectly fused to the molecule selectively binding to the protein of interest via a first linker peptide or a first chemical linker.

43. The chimera according to claim 38, characterized in that the lysosome-targeting structural unit is directly fused to the cell-penetrating peptide, or fused to the cell-penetrating peptide via a second linker peptide or a second chemical linker.

44. The chimera according to claim 42, characterized in that the amino acid sequence of the first or second linker peptide is independently (Leu-Pro-Glu-Thr)ₓ-(Gly_{y1}-Ser_{y2})_{z}, in which x=0 or 1, y1=3, 4, or 5, y2=0 or 1, and z=1, 2, or 3.

45. The chimera according to claim 44, characterized in that the first or second linker peptide is at least one independently selected from the group consisting of the following amino acid sequences: Leu-Pro-Glu-Thr-Gly-Gly-Gly (SEQ ID NO: 31), Gly-Gly-Gly, Gly-Gly-Gly-Gly-Gly (SEQ ID NO: 32), Gly-Gly-Gly-Gly-Ser (SEQ ID NO: 33), Gly-Gly-Gly-Gly-Ser-Gly-Gly-Gly-Gly-Ser (SEQ ID NO: 34), and Gly-Gly-Gly-Gly-Ser-Gly-Gly-Gly-Gly-Ser-Gly-Gly-Gly-Gly-Ser (SEQ ID NO: 35).

46. The chimera according to claim 37, characterized in that the molecule selectively binding to the protein of interest comprises a polypeptide, a protein, a nucleic acid, a nanoparticle, or a small molecule compound;
preferably, the polypeptide is glycosylated or non-glycosylated, or consists of natural amino acids and/or unnatural amino acids.

47. The chimera according to claim 46, characterized in that the signal sequence mediating the transport of the protein of interest to the lysosome is fused to the polypeptide at at least one of the C-terminus of the polypeptide, the N-terminus of the polypeptide, and an amino acid side chain group of the polypeptide;
preferably, the amino acid side chain group comprises a side chain group of a natural amino acid or a side chain group of an unnatural amino acid;
preferably, the side chain group of a natural amino acid comprises an amino group and a thiol group; and/or the side chain group of an unnatural amino acid comprises an azido group, alkyne group, aldehyde group, ketone group, fluoro sulfonate, chloro group, bromo group, or iodo group.

48. The chimera according to claim 46, characterized in that the protein is an antibody;
preferably, the antibody comprises a polyclonal antibody, a monoclonal antibody, a monospecific antibody, a bispecific antibody, a multispecific antibody, a chimeric antibody, a humanized antibody, a human antibody, or an antigen-binding fragment;
preferably, the antigen-binding fragment is a Fab, a Fab′, a F(ab′)₂, a Fv, a dsFv, a scFv, a sc(Fv)₂, or a VHH.

49. The chimera according to claim 48, characterized in that the signal sequence mediating the transport of the protein of interest to the lysosome is fused to the protein at at least one of the C-terminus of the heavy chain of the antibody, the N-terminus of the heavy chain of the antibody, the C-terminus of the light chain of the antibody, the N-terminus of the light chain of the antibody, an amino acid side chain group of the antibody, the C-terminus of the antigen-binding fragment, the N-terminus of the antigen-binding fragment, and an amino acid side chain group of the antigen-binding fragment.

50. The chimera according to claim 37, characterized in that the signal sequence mediating the transport of the protein of interest to the lysosome is fused to the molecule selectively binding to the protein of interest by a method comprising at least one of chemical conjugation, enzymatic catalysis, and genetic recombination.

51. The chimera according to claim 37, characterized in that the protein of interest is at least one of a cell surface protein, an intracellular protein, and an extracellular protein.

52. The chimera according to claim 37, characterized in that the protein of interest is at least one of a disease-associated protein of interest and an immune checkpoint molecule.

53. The chimera according to claim 52, characterized in that the disease comprises at least one of a cancer, an inflammation-related disease, an immune-related disease, a viral infection, a metabolic disease, and a neurodegenerative disease.

54. The chimera according to claim 37, characterized in that the protein of interest is at least one of an internalizable or non-internalizable receptor, a ligand of a receptor, a cytokine, a hormone, a secreted protein, an antibody, a protein carrier, and an enzyme.

55. The chimera according to claim 37, characterized in that the chimera is used for targeted protein degradation.

56. A nucleic acid molecule comprising a nucleotide sequence encoding the chimera according to claim 37.
